(19) United States
(12) Patent Application Publication
(10) Pub. No.: US 2025/0146069 A1
(43) Pub. Date: May 8, 2025

(54) ANCHORED PRIMARY NUCLEIC ACID PROBES AND METHODS THEREOF; RIBONUCLEASE-INSENSITIVE METHODS FOR DETERMINING CELLULAR NUCLEIC ACID IN A BIOLOGICAL SAMPLE

(71) Applicant: Vizgen, Inc., Cambridge, MA (US)

(72) Inventors: Jiang He, Arlington, MA (US); Lizhi He, Boston, MA (US)

(21) Appl. No.: 18/938,690

(22) Filed: Nov. 6, 2024

Related U.S. Application Data
(60) Provisional application No. 63/596,541, filed on Nov. 6, 2023.

(51) Int. Cl. C12Q 1/6874; C12Q 1/6806; C12Q 1/6841; C12Q 1/6876; G01N 21/64

(52) U.S. Cl. CPC C12Q 1/6874; C12Q 1/6806; C12Q 1/6841; C12Q 1/6876; G01N 21/6428; G01N 21/6456; G01N 2021/6432; G01N 2021/6441

(57) ABSTRACT

The present disclosure is generally directed to anchoring primary nucleic acid probes in a polymer matrix, which are configured to hybridize to cellular nucleic acid, embedding in a polymer matrix, clearing cellular components including the cellular nucleic acid and imaging. The disclosure describes methods for imaging cellular RNA in an RNase insensitive method.

### CROSS-REFERENCE TO RELATED APPLICATIONS

This application claims the benefit of priority from U.S. Provisional Application No. 63/596,541, filed Nov. 6, 2023 (now pending). The specification of the foregoing application is incorporated herein by reference in its entirety.

### FIELD OF THE DISCLOSURE

This application relates generally to the field of in situ imaging, and in particular, relates to methods of determining nucleic acid targets within tissue samples.

### BACKGROUND OF THE DISCLOSURE

To accurately profile in situ gene expression in a biological sample, a spatial transcriptomics technique with high detection efficiency and spatial resolution is required. In situ single cell transcriptomic imaging technology, such as Multiplexed Error-Robust Fluorescence In Situ Hybridization (MERFISH), enables the direct profiling of the spatial organization of intact tissue with subcellular resolution. Through combinatorial labeling and sequential imaging, different RNA species are imaged by fluorescence microscopy to generate a specific pattern of fluorescent ON and OFF signal, an optical barcode that can be used to resolve the location and quantity of different RNA species. This spatially resolved RNA-profiling data gives a physical picture for the cell or tissue of interest, which can help elucidate the intricate interplay between different cell types in complex biological systems.

However, RNA species are vulnerable to chemical insults from endogenous and exogenous sources, and the ubiquitous presence of ribonuclease (RNase) in the environment poses a great challenge to RNA imaging. This challenge is particularly concerning for RNA imaging technologies, such as MERFISH, that require prolonged imaging time up to several days. Reagents such as RNase inhibitors can be used to prevent RNA degradation. However, chemical RNase inhibitors are costly, and they need to be replenished during RNA imaging as their activity drops overtime. Thereby, there is a strong need to develop a RNase-insensitive method to analyze the RNA in biological samples.

### SUMMARY OF THE DISCLOSURE

Described herein are methods and reagents thereof for in situ single-cell transcriptomic analysis from biological samples.

In certain embodiments provided herein are methods for anchoring a primary nucleic acid probe within a matrix and clearing cellular components. In embodiments, the method comprises contacting a biological sample with a primary nucleic acid probe and an anchoring agent, wherein the primary probe comprises an acrydite moiety and hybridizes with a target nucleic acid and the anchoring agent forms a covalent bond with the primary nucleic acid probe; embedding the biological sample in a polymer matrix wherein the primary nucleic acid probe and the anchoring agent each form a covalent bond with the polymer matrix; and clearing cellular components from the polymer matrix wherein the primary nucleic acid probe hybridized to the target nucleic acid remains anchored in the polymer matrix to form a matrix anchored primary nucleic acid probe sample.

In certain embodiments provided herein is a method for imaging target nucleic acid within a matrix and clearing cellular components. In embodiments, the method comprises contacting a biological sample with a primary nucleic acid probe and an anchoring agent, wherein the primary probe comprises an acrydite moiety and hybridizes with a target nucleic acid and the anchoring agent forms a covalent bond with the primary nucleic acid probe; embedding the biological sample in a polymer matrix wherein the primary nucleic acid probe and the anchoring agent each form a covalent bond with the polymer matrix; clearing non-target cellular components from the polymer matrix wherein the primary nucleic acid probe hybridized to the target nucleic acid remains anchored in the polymer matrix to form a matrix anchored primary nucleic acid probe sample; and, contacting the anchored primary nucleic acid probe with a plurality of secondary nucleic acid probes comprising a fluorescent label and a recognition sequence that hybridizes to a sequence of the primary nucleic acid probe and imaging the target nucleic acids.

In certain other embodiments provided herein is a method for anchoring a primary nucleic acid probe within a matrix and clearing cellular components, wherein the cellular nucleic acid is first anchored in a polymer matrix. In embodiments, the method comprises contacting a biological sample with a first and/or second anchoring agent, wherein the first anchoring agent forms a covalent bond with the target nucleic acid and the second anchoring agent comprises an oligonucleotide that hybridizes with the target nucleic acid and wherein the first anchoring agent and the second anchoring agent each comprise an acrydite moiety that covalently binds to the polymer matrix; embedding the biological sample in a polymer matrix wherein the first and second anchoring agents each form a covalent bond with the polymer matrix; clearing the cellular components from the polymer matrix wherein the target nucleic acid remains anchored in the polymer matrix; contacting the anchored target nucleic acid with a primary nucleic acid probe and the first anchoring agent, wherein the primary nucleic acid probe comprises an acrydite moiety and hybridizes with the target nucleic acid and the anchoring agent forms a covalent bond with the primary nucleic acid probe; and, embedding the sample in a polymer matrix wherein the primary nucleic acid probe and the anchoring agent each form a covalent bond with the polymer matrix to form a matrix anchored primary nucleic acid probe sample. In further embodiments, the anchored primary probes may be imaged.

In certain embodiments provided herein is a primary nucleic acid probe comprising: an acrydite moiety and a target sequence configured to hybridize with a target nucleic acid in a biological sample. In certain embodiments the primary nucleic acid probes are present in a pool wherein each of the primary nucleic acid probes comprises a target sequence, one or more read sequences and an acrydite moiety, wherein each pool of primary nucleic acid probes is configured to hybridize to a distinct RNA species in a sample and each pool of primary probes encode a N-bit code that was assigned to each distinct RNA species, wherein each assigned N-bit code is a valid codeword.

Provided in certain embodiments, is a method for anchoring primary probe pools within a polymer matrix and clearing cellular components. In embodiments, the methods comprise contacting a biological sample comprising a plurality of distinct nucleic acid species with a plurality of primary nucleic acid probe pools each of the primary nucleic acid probes comprising a target sequence, one or more read sequences and one or more moieties that covalently binds to a polymer matrix, wherein each pool of nucleic acid probes hybridize to a distinct nucleic acid species and each pool of probes encode a N-bit code that was assigned to each distinct nucleic acid species; polymerizing a polymer matrix within the biological sample; anchoring the nucleic acid probes to the polymer matrix wherein the primary nucleic acid probes covalently bind the polymer matrix; and, clearing cellular components from the polymer matrix wherein the primary nucleic acid probes of the distinct nucleic acid species remain anchored in the polymer matrix.

In embodiments, the methods further comprise imaging the primary nucleic acid probe pools comprising contacting the anchored primary nucleic acid probes with a plurality of fluorescent readout probes that hybridize to the read sequences of the primary nucleic probes; imaging the readout probes bound to the primary nucleic acid probes; and, repeating steps a) and b) in one or more sequential hybridization and imaging rounds until all N positions in the code have been imaged providing an imaged code corresponding to each distinct nucleic acid species in a spatial organization.

### BRIEF DESCRIPTION OF THE DRAWINGS

FIG. 1 shows the workflow of Protocol A (methods of this disclosure) and Protocol B (methods of this disclosure). See Example 4 and 5.

FIG. 2 shows generation of a primary probe pool comprising a 5′ acrydite moiety. See Example 1.

FIG. 3 shows (FIG. 3A) spatial distribution of imaged gene transcripts from U2OS cells and (FIG. 3B) a bar graph with transcripts per cell using anchored primary probes (“modified gene panel”) compared to unanchored primary probes (“control gene panel”) with and without RNase treatment to remove the cellular RNA that was hybridized to the primary probes. See Example 2.

FIG. 4 shows (FIG. 4A) spatial distribution of four imaged gene transcripts from a mouse brain tissue sample and (FIG. 4B) shows a bar graph with transcripts per field of view (FOV) using anchored primary probes (“modified gene panel”) compared to unanchored primary probes (“control gene panel”) with and without RNase treatment to remove the cellular RNA that was hybridized to the primary probes. See Example 2.

FIG. 5 shows a bar graph with transcripts per field of view (FOV) in a mouse brain tissue sample using anchored primary probes and an anchoring agent followed by an RNase treatment (“Anchoring Agent 1 (or 2) with RNase”) compared to unanchored primary probes (“Ctrl”). See Example 3.A.

FIG. 6 shows a bar graph with transcripts per field of view (FOV) in a mouse brain tissue sample using anchored primary probes and four different test conditions; “RNA anchoring” where the cellular RNA was first anchored to a polymer matrix; “RNase treatment” applied to anchored primary probes; “Anchoring Agent 2 modification” where the anchoring agent was added to the primary probe in situ and polymerized in a polymer matrix; and “second gel” which was added to samples that had more than one anchoring agent step or reactive group that needed to be polymerized with the polymer matrix. See Example 3B.

FIG. 7 shows a bar graph of transcripts per cell from U2OS cells under various conditions, including the “double modification” where the primary probes were anchored via an acrydite moiety and an anchoring agent such as alkylating agent derivatized with an acrydite moiety added to the primary probes in situ. See Example 4.

FIG. 8 shows correlation data with bulk RNAseq and correlation between the datasets from the data of FIG. 7. See Example 4.

FIG. 9 shows a bar graph with transcripts per field of view (FOV) in a mouse brain tissue sample following the work flow of FIG. 1 (“A” and “B”) compared to unanchored primary probes (“Protocol C”). See Example 5.

FIG. 10 shows correlation data with bulk RNAseq from the data of FIG. 9. See Example 5.

### DETAILED DESCRIPTION OF THE DISCLOSURE

The present disclosure generally relates to anchoring primary nucleic acid probes in a biological sample that is embedded in a polymer matrix. Immobilizing or covalently binding the primary nucleic acid probes in polymer matrix embedded biological sample provides an RNase-insensitive method for imaging cellular nucleic acid. In embodiments, the primary probes comprise a target sequence that hybridizes to a cellular nucleic acid target and a moiety that forms a covalent bond with a polymer matrix. In embodiments, the primary nucleic acid acrydite moiety, and amine moiety or an or allyl moiety. One of skill in the art understands the moiety is selected based on preferred chemistry and the selection of the polymer matrix (e.g., polyacrylamide gel). In embodiments, the biological sample is a cell suspension, a cell from cell culture, fresh or frozen tissue sample, or FFPE (formalin fixed paraffin embedded) tissue samples. The present disclosure also provides preparation of tissue samples that allows in situ single-cell transcriptomic imaging (e.g., MERFISH, smFISH) to detect nucleic acid targets in the samples. The methods of the present disclosure can be used for the preparation of gene expression profiles of tissue samples. Other aspects are generally directed to systems or kits involving such methods or the like.

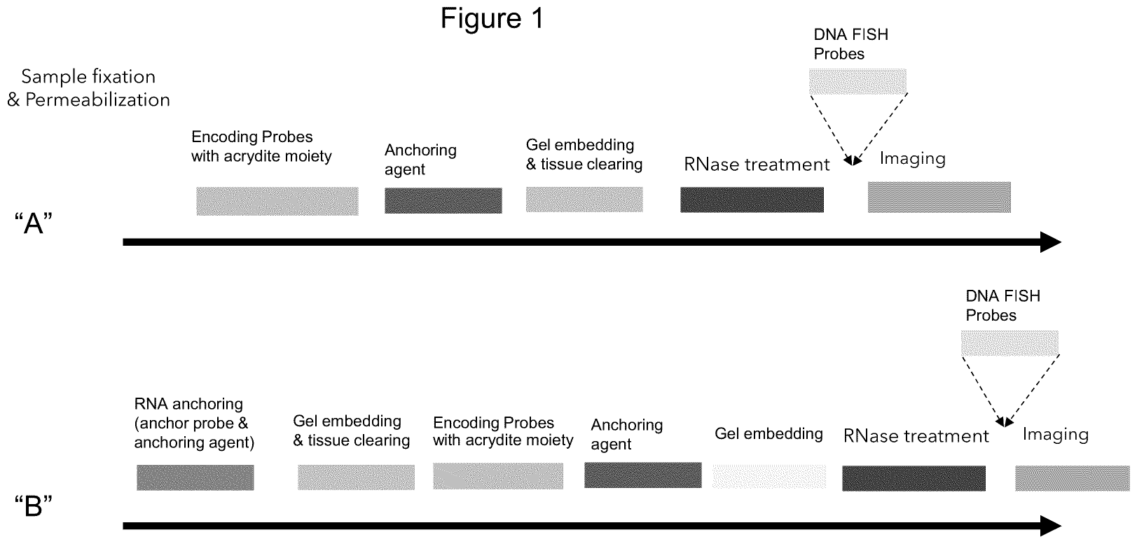

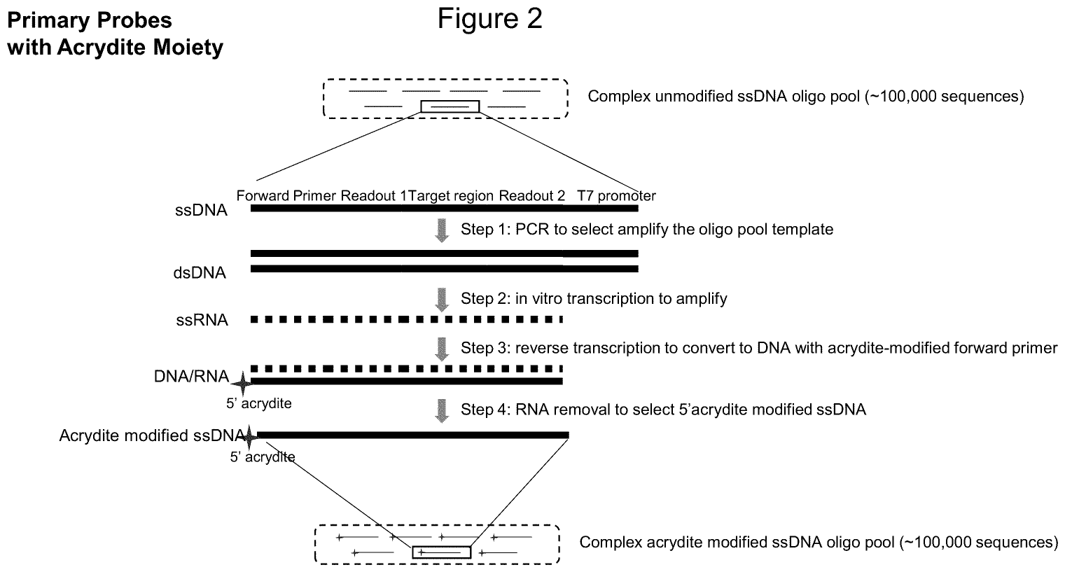

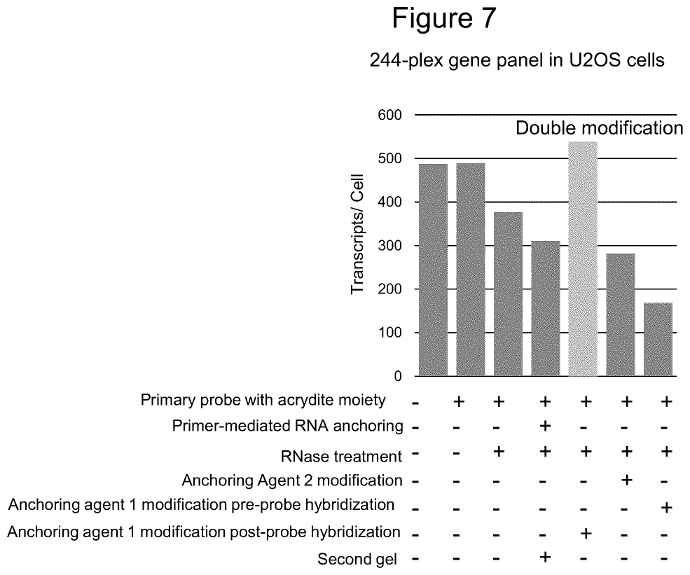

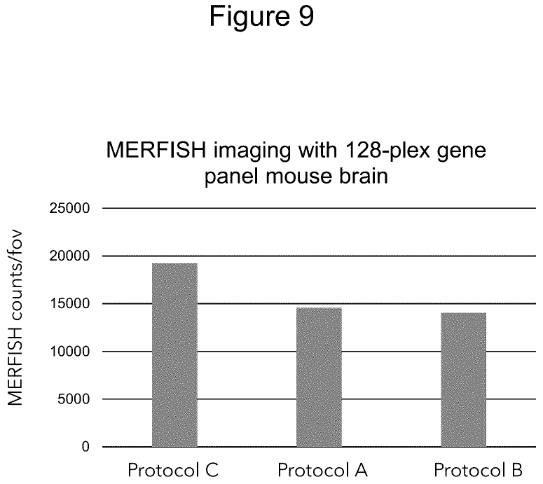

Methods of this disclosure comprise a method to determine the nucleic acids in biological samples resistant to RNase degradation by utilizing oligo probes (“primary nucleic acid probes”) containing target regions that can hybridize to nucleic acids in a cell or tissue, a moiety that forms a covalent bond with a polymer matrix (e.g., 5′ labeled acrydite moiety) and optionally read sequences that can bind with fluorescently labeled readout probes. See FIG. 2 and Example 1. These primary nucleic acid probes comprising an acrydite moiety are added to a biological sample where they hybridize to cellular nucleic acid target, the biological sample is embedded with a polymer matrix wherein the acrydite moiety of the primary probe covalently binds with the polymer matrix. Once the primary probes are anchored or immobilized in the polymer matrix, cellular components, including the cellular nucleic acid target hybridized to the primary probes, can be cleared and removed from the sample. Subsequently secondary probes and/or labels can be added and imaging completed. See Example 3. In certain embodiments, an anchoring agent is added to the primary probe in situ, wherein the anchoring agent forms a covalently bond with the primary probe and then with the polymer matrix when the biological sample is embedded with the polymer matrix. See Example 4, FIG. 1 (“Protocol A”) and FIG. 7 “double modification”. In certain other embodiments, when fragmentation of the nucleic acid, especially mRNA, is suspected (e.g., FFPE samples), the cellular nucleic acid is anchored in a polymer matrix before the primary probes comprising a moiety that forms a covalent bond with the polymer matrix. See Example 5, FIG. 1 (“Protocol B”) and FIG. 9. In embodiments, anchoring the cellular nucleic acid target comprises contacting the biological sample with a first and/or second anchoring agent, wherein the first anchoring agent forms a covalent bond with the target nucleic acid and the second anchoring agent comprises an oligonucleotide that hybridizes with the target nucleic acid.

In embodiments, the biological sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel. In embodiments, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

Examples of suitable polymers include polyacrylamide and agarose. In some cases, the polymer is a gel or a hydrogel. A variety of polymers could be used in various embodiments that involve chemical cross links between gel subunits, including but not limited to acrylic acid, acrylamide, ethylene glycol diacrylate, ethylene glycol dimethacrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alginate, chitosan, gum Arabic, gelatin, and agarose.

In certain embodiments are methods for anchoring a primary nucleic acid probe within a matrix and clearing cellular components, which can then be used for downstream imaging of the nucleic acid targets. In embodiments, the methods comprise contacting biological sample with a primary nucleic acid probe and an anchoring agent, wherein the primary probe comprises an acrydite moiety and hybridizes with a target nucleic acid and the anchoring agent forms a covalent bond with the primary nucleic acid probe; embedding the biological sample in a polymer matrix wherein the primary nucleic acid probe and the anchoring agent each form a covalent bond with the polymer matrix; and clearing cellular components from the polymer matrix wherein the primary nucleic acid probe hybridized to the target nucleic acid remains anchored in the polymer matrix to form a matrix anchored primary nucleic acid probe sample.

In certain other embodiments, the methods comprising contacting a biological sample comprising a plurality of distinct nucleic acid species with a plurality of primary nucleic acid probe pools each of the primary nucleic acid probes comprising a target sequence, one or more read sequences and one or more moieties that covalently binds to a polymer matrix, wherein each pool of nucleic acid probes hybridize to a distinct nucleic acid species and each pool of probes encode a N-bit code that was assigned to each distinct nucleic acid species; polymerizing a polymer matrix within the biological sample; anchoring the nucleic acid probes to the polymer matrix wherein the primary nucleic acid probes covalently bind the polymer matrix; and, clearing cellular components from the polymer matrix wherein the primary nucleic acid probes of the distinct nucleic acid species remain anchored in the polymer matrix. In embodiments, the method further comprises imaging the anchored primary nucleic acid probes. In certain embodiments, the methods comprise contacting the anchored primary nucleic acid probes with a plurality of fluorescent readout probes that hybridize to the read sequences of the primary nucleic probes; imaging the readout probes bound to the primary nucleic acid probes; and, repeating steps a) and b) in one or more sequential hybridization and imaging rounds until all N positions in the code have been imaged providing an imaged code corresponding to each distinct nucleic acid species in a spatial organization.

In certain embodiments, the target nucleic acid is RNA, in particular mRNA, wherein the methods comprise contacting a biological sample suspected of containing fragments RNA (e.g., formalin fixed paraffin embedded (FFPE) tissue sample) with at least two anchoring agents, wherein the first anchoring agent comprises an alkylating agent that forms a covalent bond with the target nucleic acid and the second anchoring agent comprises a polyT sequence that is complementary and hybridizes to the target RNA and wherein the first anchoring agent and the second anchoring agent each comprise an acrydite moiety that covalently binds to the matrix; embedding the biological sample in a polymer matrix wherein the first and second anchoring agents each form a covalent bond with the polymer matrix; and, clearing the cellular components from the polymer matrix wherein the target RNA remains anchored in the polymer matrix. The primary probes of this disclosure are then added to the anchored RNA and a send gel embedding step is performed to anchor the primary probe to the polymer gel rendering the imaging RNase insensitive.

In certain embodiments, FFPE tissue sections are prepared for transcriptome analysis (e.g., RNA transcripts) comprising the steps of deparaffinization, ethanol rehydration, antigen retrieval, followed by the addition of at least two anchoring agents (e.g., such as two functionally different or distinct anchoring agents) to the tissue sample, wherein a first anchoring agent forms a covalent bond with the target nucleic acid and a second anchoring agent (also referred to herein as an “anchor probe”) comprises an oligonucleotide that hybridizes with the target nucleic acid. In this way, the target nucleic acid has been functionalized with two separate anchoring agents to form an anchor treated tissue sample. This anchoring treatment step improves immobilization of the target nucleic acid within a gel or polymer matrix, wherein mRNA, in particular, can become fragmented during the FFPE process of fixing tissue sections. Each of these anchoring agents further comprise a chemical moiety (e.g., reactive group) that can form a covalent bond with a polymer matrix either during (polymerization) or after the polymer matrix has formed. Accordingly, after the at least two anchoring agents are added to the tissue sample the sample is then embedded in a polymer matrix wherein the first and second anchoring agents each form a covalent bond with the polymer matrix. In this way, the target nucleic acids are immobilized in the polymer gel matrix. In certain embodiments, the polymer matrix is a polyacrylamide gel and the first and second anchoring agents each comprise a reactive group that will form a covalent bond with acrylamide (e.g., acrydite).

After the gel embedding step, the cellular components (also referred to herein as a tissue clearing or digestion step) are removed using reagents and methods known in the art (e.g., protease digestion). In embodiments, this clearing step, in combination with the use of at least two anchoring agents when starting with FFPE tissue sample, removes the protein crosslinking induced by the formalin fixing process exposing target nucleic acid to a complimentary primary oligonucleotide probe designed to hybridize to a target sequence in the anchored nucleic acid.

In embodiments, provided herein are methods for imaging a nucleic acid target within a matrix and clearing cellular components. The method comprises contacting a biological sample with a primary nucleic acid probe and an anchoring agent, wherein the primary probe comprises an acrydite moiety and hybridizes with a target nucleic acid and the anchoring agent forms a covalent bond with the primary nucleic acid probe; embedding the biological sample in a polymer matrix wherein the primary nucleic acid probe and the anchoring agent each form a covalent bond with the polymer matrix; clearing cellular components from the polymer matrix wherein the primary nucleic acid probe hybridized to the target nucleic acid remains anchored in the polymer matrix to form a matrix anchored primary nucleic acid probe sample; and, contacting the anchored primary nucleic acid probe with a plurality of secondary nucleic acid probes comprising a fluorescent label and a recognition sequence that hybridizes to a sequence of the primary nucleic acid probe and imaging the target nucleic acids. In certain embodiments the methods include contacting nucleic acid targets (e.g., RNA transcripts) with a first and/or second anchoring agent to enhance immobilization of RNA transcript efficiency before polymer matrix embedding and addition of the modified primary probes of this disclosure. In embodiments, the methods include tissue clearing (e.g., removing non-target cellular components) prior to contacting the sample with acrydite modified primary nucleic acid probes (e.g., MERFISH probes, smFISH probes, etc.) to enhance the efficiency of primary probe binding by exposing target nucleic acid after crosslinked proteins have been cleared. Because the acrydite moiety of the primary probe needs to form a covalent bond with the polymer matrix during polymerization, a second polymer matrix is added to the sample. This renders the sample RNase insensitive, and RNase can optionally be added to the sample removing the nucleic acid target and leaving the anchored primary probes which is subsequent imaged in situ as a proxy for the cellular nucleic acid target. In certain embodiments, the methods comprise contacting the RNA transcripts (e.g., nucleic acid target) with a first and/or second anchoring agents wherein a first anchoring agent forms a covalent bond with the target nucleic acid, and the second anchoring agent (anchor probe) comprises an oligonucleotide that hybridizes with the target nucleic acid, embedding the tissue sample comprising the first and/or second anchoring agents in a gel matrix whereby the RNA transcripts are immobilized in the polymer gel matrix when the first and second anchoring agents each form a covalent bond with the polymer matrix, digesting or clearing the tissue and/or non-target cellular components followed by contacting the immobilized nucleic acid (e.g., RNA transcripts) with a plurality of acrydite modified primary probes that specifically hybridize to the immobilized target nucleic acid, adding a second polymer matrix and imaging the immobilized primary probes.

The primary probes may be, for example, MERFISH probes or smFISH probes, and may be substantially complementary to mRNA or other RNAs, for example, for transcriptome analyses. The primary oligonucleotide probes may also include signaling entities, e.g., fluorescent signaling entities, for imaging and/or analysis of the sample. In certain embodiments, a secondary oligonucleotide probe that hybridizes to the primary probe comprises an imaging moiety (e.g., fluorescent signaling entities), wherein imaging comprises adding one or more of the secondary probes. In some embodiments, the method further comprises creating codewords or barcodes based on a distribution of the bound nucleic acid probes within the sample. In embodiments, the methods comprise contacting the biological sample comprising a plurality of distinct RNA species with a plurality of primary nucleic acid probe pools each of the nucleic acid probes comprising a target sequence, one or more read sequences and an acrydite moiety that covalently binds the polymer matrix, wherein each pool of nucleic acid probes hybridize to a distinct RNA species and each pool of probes encode a N-bit code that was assigned to each distinct RNA species, wherein each assigned N-bit code is a valid codeword with a Hamming distance of 2 between valid codewords. The read sequences corresponded to a bit value of 1, wherein each subsequent round of imaging produces a 0 (when no labeled readout probe binds a read sequence of the primary probe pool) or a 1 (when a readout probe binds or hybridizes to a read sequence) and in this way an N-bit codeword is decoded when all rounds of imaging are complete.

In embodiments, the method comprises contacting the anchored primary nucleic acid probes with a plurality of readout probes comprising a fluorescent label, wherein the readout probes hybridize to the read sequences of the primary nucleic acid probes; imaging the readout probes bound to the primary nucleic acid probes; and, repeating steps in one or more sequential hybridization and imaging rounds until all N positions in the N-bit code have been imaged providing an imaged codeword corresponding to each distinct RNA species in a spatial organization. In embodiments, the valid codewords have a Hamming distance equal to or greater than 4 between each of the valid codewords. In this way, an error (e.g., a 0 incorrectly read as a 1) can be identified and the identified codeword matched with a valid codeword when there is a code space of at least 4 between each of the assigned valid code words. In this scenario, if an imaged codeword contains more than one error, the codeword is discarded instead of matched to a valid codeword. In embodiments, the N-bit code has a Hamming weight of at least 4, meaning there are at least four 1s in the code which correspond to four distinct read sequences in each of the primary probe pools. In embodiments, each primary nucleic acid probe pool comprises at least 10 different primary nucleic acid probes. In certain embodiments, the primary nucleic acid probes comprise a target sequence, with an average length of between 10 and 200 nucleotides, that hybridize the distinct RNA species.

### Biological Samples

As used herein, “biological sample” herein refers to a collection of cells obtained from a biological source (e.g., cell culture or tissue from a subject). The tissue may contain nucleated cells with chromosomal material. The source of the tissue sample may be solid tissue, as from a fresh, frozen, fixed, FFPE, and/or preserved organ or tissue sample, or biopsy, or aspirate, or blood or any blood constituents, or bodily fluids, such as cerebral spinal fluid, amniotic fluid, peritoneal fluid, or interstitial fluid, or cells from any time in gestation or development of the subject. The tissue sample may also be primary or cultured cells or cell lines, or culture tissues. The tissue sample may contain compounds which are not naturally intermixed with the tissue in nature, such as preservatives, anticoagulants, buffers, fixatives, nutrients, antibiotics, or the like. In some embodiments of the invention, the tissue sample is non-hematologic tissue (i.e., not blood or bone marrow tissue). In embodiments, the biological sample is fresh frozen or fixed frozen tissue sample. In embodiments, the tissue sample used in the present methods is a formalin-fixed paraffin embedded tissue sample.

In embodiments, nucleic acid fragmentation can be evaluated and determined using methods well known in the art. For example, to evaluate sample quality for in situ hybridization, it is it is informative to determine the RNA quality of a tissue block using RNA Integrity Number (RIN) or DV200 values via commercially available instruments such as the BioAnalyzer or TapeStation platforms. Briefly, RNA from the samples are extracted first and measured on either BioAnalyzer or TapeStation. RIN is expressed in values that range from 1-10, where 1 indicates a sample has shorter and more degraded RNA whereas 10 reflects longer and less degraded RNA. Higher RIN scores will have more intact 18S and 28S RNAs. DV200 reflects the percentage of RNA fragments greater than 200 nucleotides in length in tissue samples. Tissues with lower DV200 percentages have shorter and more degraded RNA; conversely, a higher percentage indicates longer, and less degraded RNA molecules present in the tissue.

In some embodiments, the tissue sample is a tissue section, a clinical smear, or a cultured cell or tissue. In some embodiments, the tissue sample comprises a tissue section. As used herein, “section” of a tissue sample herein refers to a single part or piece of a tissue sample, for example, a thin slice of tissue or cells cut from a tissue sample. It is understood that multiple sections of tissue samples may be taken and subjected to analysis according to the present invention. In some embodiments, the selected portion or section of tissue comprises a homogeneous population of cells. In some embodiments, the selected portion or section of tissue comprises a heterogeneous population of cells. In some embodiments, the selected portion comprises a region of tissue, e.g., the lumen as a non-limiting example. The selected portion can be as small as one cell or two cells, or could represent many thousands of cells, for example.

Any tissue sample from the subject may be used. Examples of tissue samples that may be used include, but are not limited to, breast, prostate, ovary, colon, lung, endometrium, stomach, salivary gland, or pancreas. The tissue sample can be obtained by a variety of procedures including, but not limited to, surgical excision, aspiration, or biopsy. The tissue may be fresh frozen, fixed frozen or FFPE.

In some embodiments, the tissue section is a tissue section of brain, adrenal glands, colon, small intestines, stomach, heart, liver, skin, kidney, lung, pancreas, testis, ovary, prostate, uterus, thyroid, and spleen of a mammal (e.g., human or mouse). The methods of the present disclosure may be applied to any type of tissue, including, for example, cancer tissue (including from any cancer). In some embodiments, the tissue section is from a solid tumor. In some embodiments, the tissue sample is from mouse small intestine. In some embodiments, the tissue sample is from mouse brain. In some embodiments, the tissue sample is from human liver cancer. In some embodiments, the tissue sample is from human kidney. In some embodiments, the tissue sample is from human lung. In some embodiments, the tissue sample is from human ovarian cancer. In some embodiments, the tissue sample is from human uterus cancer. In some embodiments, the tissue sample is from human lung cancer.

In some embodiments, the tissue has been stored for a period of time, for example, the period of time that frozen or FFPE are stored. In some embodiments, the tissue sample is a frozen tissue sample. In some embodiments, the tissue is frozen tissue. In some embodiments, the tissue is paraffin-embedded tissue. In some embodiments, the tissue is formalin-fixed paraffin-embedded tissue.

### A. Preparation of Tissue Sample

### Obtaining and Fixing Tissue Samples

Tissue samples can be obtained from an intact organ or tissue using any methods well known to those of skill in the art, e.g., the prior methods used to prepare tissue samples for immunohistochemistry (IHC) or in situ hybridization (ISH) techniques.

For example, any intact organ or tissue may be cut into reasonably small piece(s) (the size of the cut pieces typically ranges from a few millimeters to a few centimeters) and “fixed” to preserve the positions of the nucleic acids within the sample. Techniques for fixing cells and tissues are known to those of ordinary skill in the art. Non-limiting examples of fixatives include such as formaldehyde, paraformaldehyde, glutaraldehyde, ethanol, methanol, acetone, acetic acid, or the like.

In some embodiments, the tissue sample is fixed in a solution containing an aldehyde. In some embodiments, the tissue sample is fixed in a solution containing formalin. In some embodiments, the tissue sample is paraffin embedded. In embodiments, the tissue sample is both formalin-fixed and paraffin-embedded (FFPE).

In addition to intact samples, other samples may be used. In some embodiments, the frozen-sections may be prepared by rehydrating 50 mg of frozen pulverized tissue at room temperature in phosphate-buffered saline (PBS) in a small plastic capsule; pelleting the particles by centrifugation; resuspending the particles in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections. Similarly, permanent tissue sections may be prepared involving rehydration of the 50 mg sample in a plastic microfuge tube; pelleting; resuspending in 10% formalin for a 4 hour fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

In some embodiments, the present invention may utilize standard frozen samples, such as those that are embedded in OCT and that are not pulverized, for example, including those used in standard Frozen Section hospital labs.

Tissue samples are often fixed by conventional methodology. Aldehyde fixatives such as formalin (formaldehyde) and glutaraldehyde are typically used. Tissue samples fixed using other fixation techniques, such as alcohol immersion, are also suitable. See Battifora and Kopinski, J., Histochem. Cytochem., 34:1095 (1986). One of skill in the art will appreciate that the choice of the fixative is determined by the purpose for which the tissue is to be histologically stained or otherwise analyzed. One of skill in the art will also appreciate that the length of fixation depends upon the size of the tissue sample and the fixative used.

The samples used may also be embedded in paraffin. In some embodiments, the tissue sample is fixed and embedded in paraffin or the like. In some embodiments, the tissue sample is both formalin-fixed and paraffin-embedded. In some embodiments, the formalin-fixed paraffin-embedded (FFPE) tissue block is hematoxylin and eosin (H&E) stained. As commonly known in the art, the tissue sample may be first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology. Examples of paraffin that may be used include, but are not limited to, Paraplast, Broloid, and Tissuemay. Once the tissue sample is embedded, the sample may be sectioned by a microtome or the like. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

In some embodiments, the tissue section may range from about 3 μm to about 100 μm, or any intermediate ranges therewithin. In some embodiments, the tissue section may range from about 10 μm to about 100 μm. In some embodiments, the tissue section may range from about 10 μm to about 50 μm. In some embodiments, the tissue section may range from about 10 μm to about 30 μm. In some embodiments, the tissue section may range from about 10 μm to about 15 μm. In some embodiments, the tissue section may range from about 3 μm to about 15 μm. In some embodiments, the tissue section may range from about 5 μm to about 20 μm. In some embodiments, the tissue section may range from about 15 μm to about 30 μm. In some embodiments, the tissue section may range about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, or about 20 μm. In some embodiments, the tissue section may range about 30 μm, about 40 μm, about 50 μm, about 60 μm, about 70 μm, about 80 μm, about 90 μm, or about 100 μm.

Tissue sections can be deparaffinized using methods known in the art and/or commercially available kits. The methods remove the bulk of paraffin from the sample. Various techniques are known for deparaffinizing and include, but are not limited to, washing with an organic solvent or agent to dissolve the paraffin.

Exemplar deparaffinization solvents include but are not limited to, benzene, toluene, ethylbenzene, xylenes, D-limonene, octane, and mixtures thereof. In certain embodiments, the deparaffinization solvents comprise D-limonene. These solvents are preferably of high purity, usually greater than 99%. The volume used and the number of washes necessary will depend on the size of the sample and the amount of paraffin to be removed. A sample may be washed between 1 and about 10 times, or between about two and about four times. A typical volume of organic solvent is about 500 ml for a 10 mm tissue sample.

After deparaffinization, samples may be rehydrated such as by stepwise washing with aqueous lower alcoholic solutions of decreasing concentrations. Ethanol is a preferred lower alcohol for rehydrations while other alcohols may also be used. Non-limiting examples include methanol, isopropanol, and other C₁-C₅ alcohols. The sample is alternatively vigorously mixed with alcoholic solutions followed by its removal. In some embodiments, deparaffinization and rehydration are carried out simultaneously using a reagent such as EZ-DEWAN™ (BioGenex, San Ramon, CA), for example.

In some embodiments, the concentration of alcohol is stepwise lowered. In some embodiments, the concentration range of alcohol is decreased stepwise from about 100% to about 70% in water over about three to five incremental steps. In some embodiments, the concentration range of alcohol is decreased stepwise over three incremental steps with 100%, 90%, and 70% respectively.

In some embodiments of the present disclosure, the samples may be pretreated, such as to facilitate directly or indirectly the methods of the invention. In some embodiments, pretreatment of the tissue increases availability of the target nucleic acid or other targets (e.g., for cell morphology staining). Pretreatments for making targets available (e.g., “antigen retrieval” that retrieves or unmasks the biological markers of interest). An extensive review of antigen retrieval may be found in Shi et al. 1997, J Histochem Cytochem, 45 (3): 327. Antigen retrieval includes a variety of methods by which the availability of the target for interaction with a specific detection reagent is maximized.

The most common techniques are protease-induced epitope retrieval (PIER) or heat induced epitope retrieval (HIER). Protease-induced epitope retrieval (PIER) may employ enzymes such as proteinase K, pepsin, trypsin, protease, and any subtypes thereof, in an appropriate buffer to restore the epitope for antibody binding. Heat-induced epitope retrieval (HIER) may employ heat to reverse some cross-links and allow the restoration of epitopes. Citrate buffers, Tris, and EDTA base may be employed as exemplary heat-induced reagents in appropriately pH stabilized manner (e.g., 10 mM sodium citrate, 6.0 pH; 1 mM EDTA, pH 8.0; 10 mM Tris base, 1 mM EDTA solution, 0.05% Tween 20, pH 9.0). Detergents (e.g., Tween 20) may be added to the HIER buffer to increase the epitope retrieval. In certain aspects, many proprietary formulations may be available for the PIER or HIER mediate antigen retrieval.

Selective staining may be conducted on a tissue section for detection of biological markers and identification of cell types (e.g., nuclear and/or cell morphology stains). To facilitate the specific recognition of biological markers in fixed tissue (e.g., FFPE tissue sample post-deparaffinization and rehydration), it is often necessary to retrieve or unmask the biological markers of interest, through “antigen retrieval” (also called epitope retrieval or antigen unmasking)

In certain embodiments, cellular nucleic acid targets are immobilized in a polymer matrix prior to addition of the primary probe comprising a moiety that forms a covalent bond with the polymer matrix. See FIG. 1 “B”.

It is understood that immobilization of the cellular nucleic acid is a multi-step process, wherein a first and/or second anchoring agents are first added to the tissue sample and a covalent bond is formed between the first anchoring agent and target nucleic acid (as disclosed herein for the first anchor agent of the methods) and the second anchoring agent comprises an oligonucleotide that hybridizes with the target nucleic acid (as disclosed herein for the anchor probe or second anchoring agent of the methods) followed by contact with the polymer matrix wherein both the first and second anchoring agents form covalent bonds with the polymer gel. That entire process immobilizes the target nucleic acid in the polymer gel matrix.

In embodiments, the target nucleic acid-anchoring agents react to form a covalent bond with the polymer gel before, during or after formation of the polymer matrix.

### B. Anchoring Agents

In embodiments, a first and/or second anchoring agents are provided for immobilization of the target nucleic acid (e.g., RNA transcripts) to a polymer matrix, as discussed below. Also, as described below, the first anchoring agent is optionally added with the primary probe to further anchor the primary probes in the polymer matrix. In this way, two steps using anchoring agents; first when the cellular nucleic acid is anchored in the polymer matrix, and second to aid in the anchoring of the primary probes in the polymer matrix.

In one embodiment, a first anchoring agent is functionalized to comprise a first chemical moiety or reactive group that will form a covalent bond with the target nucleic acid, and a second chemical moiety or reactive group that will form a covalent bond with the polymer gel matrix. In another embodiment, the second anchoring agent comprises an oligonucleotide that hybridizes with the target nucleic acid and a chemical moiety or reactive group that will form a covalent bond with the polymer gel matrix. In certain embodiments, the chemical moiety or reactive group of the second anchoring agent is the same or different as the second chemical moiety of the first anchoring agent. In certain embodiments, the second anchoring agent is also referred to herein as an anchor probe due to the oligonucleotide that hybridizes to the target nucleic acid. In embodiments, the oligonucleotide portion of the second anchoring agent comprises a poly-T (thymine residues) for hybridizing with the poly-A tail of an mRNA transcript.

In some embodiments, the anchor probes may contain sequences complementary to the desired (target) nucleic acid species, e.g., binding to them via base pairing (hybridizing). In embodiments, anchor probes, comprise a chemical moiety or reactive group able to polymerize (e.g., covalent bonding) with a polymer gel matrix.

In one set of embodiments, the anchoring agents form a covalent bond during the polymerization process with the polymer gel matrix. For example, in the case of polyacrylamide, the anchoring agent may include an acrydite moiety that can polymerize and become incorporated into the polymer. In certain embodiments, the second anchoring agent or anchor probe comprises an oligonucleotide (poly-T) that hybridizes with the poly-A tail of mRNA and an acrydite moiety that forms a covalent bond with polyacrylamide, wherein the gel embedding step utilizes polyacrylamide.

The anchoring agents may also contain a portion that can interact with and bind to nucleic acid molecules, or other molecules in which immobilization is desired, e.g., proteins or lipids, other desired targets, etc. The immobilization may be covalent or non-covalent. For example, to immobilize a cellular nucleic acid, the anchoring agents may comprise a nucleic acid comprising an acrydite portion (e.g., at the 5′ end, the 3′ end, an internal base, etc.) and a nucleic acid sequence substantially complementary to at least a portion of the target nucleic acid. For instance, the nucleic acid may be complementary to at least 5, 6, 7, 8, 9, 10, 11, 12, 13, 14, 15, 16, 17, 18, 19, or 20 or more nucleotides of the nucleic acid. In some cases, the complementarity may be exact (Watson-Crick complementarity), or there may be 1, 2, or more mismatches. In some cases, the anchoring agent can be configured to immobilize mRNA, e.g., in the case of transcriptome analysis. For instance, in one set of embodiments, the anchoring agent may contain a plurality of thymine nucleotides, e.g., sequentially, for binding to the poly-A tail of an mRNA. Thus, for example, the anchoring agent can have at least 5, 6, 7, 8, 9, 10, 11, 12, 13, 14, 15, 16, 17, 18, 19, or 20 or more consecutive thymine nucleotides (e.g., a poly-dT portion) within the anchoring agent. In some cases, at least some of the thymine nucleotides may be “locked” thymine nucleotides. These may comprise at least 20%, at least 30%, at least 40%, at least 50%, at least 60%, at least 70%, or at least 80% of these thymine nucleotides. In certain embodiments, the locked and non-locked nucleotides may alternate. Such locked thymine nucleotides may be useful, for example, to stabilize the hybridization of the poly-A tails of the mRNA with the anchoring agent.

In embodiments, the methods herein further comprise the use of another anchoring agent, referred to herein as a first anchoring agent, wherein that anchoring agent is functionalized with a first and second chemical moiety for covalent attachment to the target nucleic acid, and to the polymer gel matrix. In certain embodiments, the anchoring agent is a derivatized alkylating agent wherein the alkylating agent has been derivatized with a chemical moiety or reactive group that will form a covalent bond with the polymer gel matrix. Alkylating agents are well known in the art, and which form a covalent bond with nucleic acid, including RNA, any of which can be derivatized to form a present anchoring agent. In certain embodiments, the anchoring agent is an alkylating agent derivatized with a reactive group that forms a covalent bond with polyacrylamide. In certain embodiments, the anchoring agent is an alkylating agent that comprises an acrydite moiety. In embodiments, alkylating agents are selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In embodiments, this first anchoring agent is also used with the primary probes to provide multiple anchor sites to the polymer matrix.

In certain embodiments, the present methods use both an anchoring probe and an anchoring agent (a first and second anchoring agent) for immobilization of the target nucleic acid in the polymer gel matrix. In some embodiments, the nucleic acid targets are immobilized within the gel via both the anchor probes and the anchoring agents bound to the nucleic acid targets.

In one set of embodiments, nucleic acid molecules may be immobilized by covalent bonding. For example, in one set of embodiments, an alkylating agent may be used that covalently binds to nucleic acid molecules and contains a second chemical moiety that can be incorporated into the polyacrylamide as it is polymerized. In yet another set of embodiments, the terminal ribose in an RNA molecule may be oxidized using sodium periodate (or another oxidizing agent) to produce an aldehyde, which may be cross-linked to acrylamide, or other polymer or gel. In other embodiments, chemical agents that are able to modify bases may be used, such as aldehydes, e.g. paraformaldehyde or gluteraldehyde, alkylating agents, or succinimidyl-containing groups; chemical agents that modify the terminal phosphate, such as carboiimides, e.g., EDC (1-ethyl-3-(3-dimethylaminopropyl) carbodiimide); chemical agents that modify internal sugars, such as p-maleimido-phenyl isocyanate; or chemical agents that modify terminal sugars, such as sodium periodate. In some cases, these chemical agents can carry a second chemical moiety that can then be directly cross-linked to the gel or polymer, and/or which can be further modified with a compound that can be directly cross linked to the gel or polymer.

In yet other embodiments, a nucleic acid may be immobilized using anchor probes having substantially complementary portions to the DNA or RNA. There may be 5, 6, 7, 8, 9, 10, 12, 13, 14, 15, 20, 25, 30, 35, 40, 45, 50 or more complementary nucleotides between the anchor probe and the nucleic acid.

### C. Gel Polymer Matrix

In embodiments, the methods disclosed herein comprise immobilizing the primary probes within a polymer matrix. See FIG. 1 “A”. In certain embodiments, the method disclosed herein further comprises immobilizing the cellular nucleic acid target within a polymer gel. See FIG. 1 “B”.

The biological sample may be embedded within a matrix that immobilizes nucleic acid targets. For instance, the matrix may comprise a gel or a polymer, such as polyacrylamide. Thus, for example, acrylamide and a suitable cross-linker (e.g., N,N′-methylenebisacrylamide) can be added to the sample and polymerized to form a gel. The primary probes or anchoring agents, if present, may include a portion able to polymerize with the gel (e.g., an acrydite moiety) during the polymerization process, and nucleic acids (e.g., mRNAs containing poly-A tails) may then be able to associate with the anchor portion. In such fashion, the mRNAs may be immobilized to the polyacrylamide gel, either directly or via hybridization to the anchored primary probes. In embodiments, DNA and/or RNA molecules may be immobilized to the polyacrylamide gel using primary probes having substantially complementary portions to the DNA or RNA. In other embodiments, cellular DNA and/or RNA molecules may be physically tangled within the polyacrylamide gel, e.g., due to their length, to immobilize them to the polyacrylamide gel.

The sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel.

A variety of polymers may be used in some embodiments. In some cases, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

Examples of suitable polymers include polyacrylamide and agarose. In embodiments, the polymer is not a hydrogel and/or does not comprise polymers or monomers that swell or expand. A variety of polymers could be used in various embodiments that involve chemical cross links between gel subunits, including but not limited to acrylic acid, acrylamide, ethylene glycol diacrylate, ethylene glycol dimethacrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alginate, chitosan, gum Arabic, gelatin, and agarose.

### II. Tissue Clearing

After immobilization of either the cellular nucleic acids targets to the gel or the primary probes (if the cellular nucleic acid was not immobilized with anchoring agents), other cellular components (e.g., non-immobilized nucleic acid) within the sample may be removed or degraded.

By “clearing” a tissue sample or a “cleared” tissue sample, it is meant that the tissue sample is made substantially permeable to light, i.e., transparent, and the optical properties of the sample change to allow more light to pass through the sample. In some embodiments, about 70% or more of the light (e.g., white light, ultraviolet light or infrared light) that is used to illuminate the sample will pass through the sample and illuminate only selected cellular components (e.g., nucleic acids) therein, e.g., 75% or more of the light, 80% or more of the light, 85% or more of the light, 90% or more of the light, 95% or more of the light, 98% or more of the light, e.g. 100% of the light will pass through the specimen. Any treatment known for tissue clearing may be used to clear the tissue sample in the methods described herein, which are further discussed below.

Details of tissue clearing have been further discussed in US Patent Publ. No. 2019/0264270 published Aug. 29, 2019, entitled “Matrix imprinting and clearing,” the content of which is incorporated herein by reference in its entirety. Such clearance may include removal (e.g., physical removal) of cellular components from the sample, and/or degradation within the sample, such that they are no longer as prominent within the background. Degradation may include, for example, chemical degradation, enzymatic degradation, or the like.

In some cases, at least 50%, at least 60%, at least 70%, at least 80%, or at least 90% of the undesired components within the sample may be removed or degraded. Such clearance may include physical removal or degradation of the components (e.g., to smaller components, components that are not fluorescent, etc.). Removal or degradation of such components may decrease background fluorescence or autofluorescence within the sample during analysis.

Multiple clearance steps can also be performed in certain embodiments, e.g., to remove or degrade various undesired components.

For example, enzymes, denaturants, chelating agents, chemical agents, and the like, may break down the proteins into smaller components and/or amino acids. These smaller components may be easier to remove physically, and/or may be sufficiently small or inert such that they do not significantly affect the background. Similarly, lipids may be removed or degraded from the sample using surfactants or the like. In some cases, one or more of these are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyanante, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to remove or degrade proteins, lipids, and/or other components. For instance, a buffer solution (e.g., containing Tris or tris(hydroxymethyl) aminomethane) may be applied to the sample, then removed.

Non-limiting examples of techniques to remove or degrade RNA include RNA enzymes such as Rnase A, Rnase T, or Rnase H, or chemical agents, e.g., via alkaline hydrolysis (for example, by increasing the pH to greater than 10). Non-limiting examples of systems to remove or degrade sugars or extracellular matrix include enzymes such as chitinase, heparinases, or other glycosylases. Non-limiting examples of systems to remove or degrade lipids include enzymes such as lipidases, chemical agents such as alcohols (e.g., methanol or ethanol), or detergents such as Triton X-100 or sodium dodecyl sulfate. Many of these are readily available commercially. In this way, the background of the sample may be reduced, which may facilitate analysis of the nucleic acid probes or other desired targets, e.g., using fluorescence microscopy, or other techniques as discussed herein.

### Cellular Nucleic Acid Targets

The nucleic acid targets may be, for example, DNA, RNA, or other nucleic acids that are present in a cell within a biological sample.

In some embodiments, the nucleic acid target is RNA. The RNA may be coding and/or non-coding RNA. Non-limiting examples of RNA that may be studied within the cell include mRNA, siRNA, rRNA, miRNA, tRNA, lncRNA, snoRNAs, snRNAs, exRNAs, piRNAs, or the like.

The nucleic acids may be endogenous to the cell, or added to the cell. For instance, the nucleic acid may be viral, or artificially created. In some cases, the nucleic acid to be determined may be expressed by the cell.

In some cases, a significant portion of the nucleic acid within the cell may be studied. For instance, in some cases, enough of the RNA present within a cell may be determined so as to produce a partial or complete transcriptome of the cell. In some cases, at least 4 unique mRNA gene transcripts are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA transcript molecules produced within a cell, coding and non-coding, not just coding messenger RNA. Thus, for instance, the transcriptome may also include non-coding rRNA, tRNA, siRNA, miRNA, etc. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

The determination of one or more nucleic acids within the sample may be qualitative and/or quantitative. In addition, the determination may also be spatial, e.g., the position of the nucleic acid within the sample may be determined in two or three dimensions. In some embodiments, the positions, number, and/or concentrations of nucleic acids within the cell (or other sample) may be determined.

### A. Nucleic Acid Probes

Provided herein are primary nucleic acid probes comprising a moiety that forms a covalent bond with the polymer matrix. See FIG. 2. These primary probes are added to the biological sample, allowed to hybridize to the (anchored) cellular nucleic acid and then immobilized in the polymer gel. Once complete, cellular components can be cleared from the sample, with may be imaged using secondary nucleic acid probes (e.g., fluorescent labeled) either simultaneously or sequentially. In alternative embodiments, when the cellular nucleic acid target is immobilized first, the cellular components are cleared before the primary probes are added. In certain embodiments, an anchoring agent is added to the sample with the primary nucleic acid probe in situ, wherein the anchoring agent forms a covalent bond with the primary nucleic acid probe. In embodiments, the anchoring agent is an alkylating agent. In certain embodiments, the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In certain embodiments, the anchoring agent and the primary nucleic acid probe each comprise an acrydite moiety that covalently binds the polymer matrix. In certain embodiments, the anchoring agent is an alkylating agent derivatized with an acrydite moiety.

For instance, in one set of embodiments, the nucleic acid probes may include smFISH or MERFISH probes, such as those discussed in U.S. Pat. No. 11,098,303 or U.S. Pat. No. 10,240,146, each incorporated herein by reference in its entirety.

The primary nucleic acid probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes, e.g., as discussed below. Any suitable method may be used to introduce nucleic acid probes into a sample.

The nucleic acid probes are added to the biological sample. Certain aspects of the present invention are generally directed to nucleic acid probes that are introduced into a sample. The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target nucleic acid, in some cases specifically. When introduced into a sample, the nucleic acid probe may be able to bind to a specific target nucleic acid (e.g., an mRNA, or other nucleic acids as discussed herein). In some cases, the nucleic acid probes may be determined using signaling entities (e.g., as discussed below), and/or by using secondary nucleic acid probes able to bind to the nucleic acid probes (i.e., to primary nucleic acid probes). The determination of such nucleic acid probes is discussed in detail below.

In some cases, more than one distinct (primary) nucleic acid probe may be applied to a sample, e.g., simultaneously. For example, there may be at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes that are applied to a sample, e.g., simultaneously or sequentially.

In certain embodiments, the primary oligonucleotide probes comprise a target sequence designed to hybridize with the anchored target nucleic acid. The target sequence may be positioned anywhere within the nucleic acid probe (or primary nucleic acid probe or encoding nucleic acid probe). The target sequence may contain a region that is substantially complementary to a portion of a target nucleic acid. In some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. Typically, complementarity is determined on the basis of Watson-Crick nucleotide base pairing.

The target sequence of a (primary) nucleic acid probe may be determined with reference to a target nucleic acid suspected of being present within a sample. For example, a target nucleic acid to a protein may be determined using the protein's sequence, by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe, such as a primary nucleic acid probe, may also comprise one or more “read” sequences designed to hybridize with secondary nucleic acid probes comprising a label (e.g., fluorescent label). However, it should be understood that read sequences are not necessary in all cases. In some embodiments, the nucleic acid probe may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences. In certain embodiments, the primary oligonucleotide probes comprise one read sequence. In certain other embodiments, the primary oligonucleotide probes comprise two read sequences, which may the same or distinct from each other (e.g., meaning a secondary nucleic acid probe will not hybridize to distinct read sequences).

The read sequences, if present, may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the sample, e.g., such that the read sequences do not themselves bind to or hybridize with other nucleic acids suspected of being within the sample. In some cases, the homology may be less than 10%, less than 8%, less than 7%, less than 6%, less than 5%, less than 4%, less than 3%, less than 2%, or less than 1%. In some cases, there may be a homology of less than 20 base pairs, less than 18 base pairs, less than 15 base pairs, less than 14 base pairs, less than 13 base pairs, less than 12 base pairs, less than 11 base pairs, or less than 10 base pairs. In some cases, the base pairs are sequential.

In certain embodiments, primary oligonucleotide probes are provided as a pool of probes, wherein each pool of nucleic acid probes hybridize to a distinct target nucleic sequence (e.g., distinct RNA transcript). In embodiments each pool of probes encode, via read sequences, a N-bit binary code that was assigned to each distinct RNA transcript. In certain embodiments, the N-bit binary code has a Hamming weight of at least 2, at least 4, at least 5, at least 6, at least 7 or at least 8, wherein the Hamming weight value is the number of “1” values in the N-bit code and all other positions are “0”. In embodiments the N-bit binary code has a Hamming weight of at least 2, or at least 4, meaning the code contains two or four “1” bit values, respectively, and the other bit positions are “0”. In embodiments, the N-bit binary code has an N value of 3 to 100, with any value thereof possible. In certain embodiments, the binary code is a 4-bit binary code, a 6-bit binary code, a 8-bit binary code, a 16-bit binary code, a 36-bit binary code, a 50-bit binary code, a 54-bit binary code or a 100-bit binary code, or any combination thereof. Each position of the binary code is either a “0” or a “1”, wherein the binding of secondary probes to the read sequence determines if the hybridization read is “0”, wherein no probe binds, or a “1” wherein secondary probe bound to the read sequence of the primary probe. Sequential hybridization and imaging of the secondary read out probes is performed until each position of the N-bit binary code has been read providing a barcode or codeword for the target nucleic acid (e.g. mRNA sequence).

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2n-1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein.

In addition, the nucleic acid probes (and their corresponding, complementary sites on the encoding probes), in certain embodiments, may be made using only 2 or only 3 of the 4 bases, such as leaving out all the guanosines (Gs) or leaving out all of the cystosines, (Cs) within the probe. Sequences lacking either Gs or Cs may form very little secondary structure in certain embodiments, and can contribute to more uniform, faster hybridization.

In some embodiments, the nucleic acid probe may contain a signaling entity. It should be understood that signaling entities are not required in all cases, however; for instance, the nucleic acid probe may be determined using secondary nucleic acid probes in some embodiments, as is discussed in additional detail below. Examples of signaling entities that can be used are also discussed in more detail below.

Other components may also be present within a nucleic acid probe as well. For example, in one set of embodiments, one or more primer sequences may be present, e.g., to allow for enzymatic amplification of probes. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In addition, the components of the nucleic acid probe may be arranged in any suitable order. For instance, in one embodiment, the components may be arranged in a nucleic acid probe as: primer-read sequences-targeting sequence-read sequences-reverse primer. The “read sequences” in this structure may each contain any number (including 0) of read sequences, so long as at least one read sequence is present in the probe. Non-limiting example structures include:
primer-targeting sequence-read sequences-reverse primer,
primer-read sequences-targeting sequence-reverse primer,
targeting sequence-primer-targeting sequence-read sequences-reverse primer,
targeting sequence-primer-read sequences-targeting sequence-reverse primer,
primer-target sequence-read sequences-targeting sequence-reverse primer,
targeting sequence-primer-read sequence-reverse primer,
targeting sequence-read sequence-primer,
read sequence-targeting sequence-primer,
read sequence-primer-targeting sequence-reverse primer, etc.
   In addition, the reverse primer is optional in some embodiments, including in all of the above-described examples.

In embodiments, the 5′ end of the primary probe is modified with an acrydite moiety, or other moieties that can form a covalent bond with the polymer matrix. See FIG. 2.

### III. Detection/Imaging of Nucleic Acid Target/Probe Complex

After introduction of the primary nucleic acid probes into a sample, the primary probes are embedded in a polymer gel (as described above) wherein the primary probes for a covalent bond with the polymer matrix.

In embodiments, the nucleic acid probes may be directly determined by determining signaling entities (if present), and/or the nucleic acid probes may be determined by using one or more secondary nucleic acid probes (also referred to herein as readout probes), in accordance with certain aspects of the invention. As mentioned, in some cases, the determination may be spatial, e.g., in two or three dimensions. In addition, in some cases, the determination may be quantitative, e.g., the amount or concentration of a primary nucleic acid probe (and of a target nucleic acid) may be determined. Additionally, the secondary probes may comprise any of a variety of entities able to hybridize a nucleic acid, e.g., DNA, RNA, LNA, and/or PNA, etc., depending on the application. Signaling entities are discussed in more detail below.

A secondary nucleic acid probe may contain a recognition sequence able to bind to or hybridize with a read sequence of a primary nucleic acid probe. In some cases, the binding is specific, or the binding may be such that a recognition sequence preferentially binds to or hybridizes with only one of the read sequences that are present. The secondary nucleic acid probe may also contain one or more signaling entities. If more than one secondary nucleic acid probe is used, the signaling entities may be the same or different. In embodiments, the secondary nucleic acid probe comprises a fluorescent label and may be referred to herein as a fluorescent secondary nucleic acid probe.

The recognition sequences may be of any length, and multiple recognition sequences may be of the same or different lengths. If more than one recognition sequence is used, the recognition sequences may independently have the same or different lengths. For instance, the recognition sequence may be at least 5, at least 10, at least 15, at least 20, at least 25, at least 30, at least 35, at least 40, or at least 50 nucleotides in length. In some cases, the recognition sequence may be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30, between 20 and 40, or between 25 and 35 nucleotides, etc. In one embodiment, the recognition sequence is of the same length as the read sequence. In addition, in some cases, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a read sequence of the primary nucleic acid probe.

As mentioned, in some cases, the secondary nucleic acid probe may comprise one or more signaling entities. Examples of signaling entities are discussed in more detail below.

As discussed, in certain aspects of the invention, nucleic acid probes are used that contain various “read sequences.” For example, a population or pool of primary nucleic acid probes may contain certain “read sequences” which can bind certain of the secondary nucleic acid probes, and the locations of the primary nucleic acid probes are determined within the sample using secondary nucleic acid probes, e.g., which comprise a signaling entity. As mentioned, in some cases, a population of read sequences may be combined in various combinations to produce different nucleic acid probes, e.g., such that a relatively small number of read sequences may be used to produce a relatively large number of different nucleic acid probes.

Thus, in some cases, a population (also referred to herein as a “pool”) of primary nucleic acid probes (or other nucleic acid probes) may each contain a certain number of read sequences, some of which are shared between different primary nucleic acid probes such that the total population of primary nucleic acid probes may contain a certain number of read sequences. A population of nucleic acid probes may have any suitable number of read sequences. For example, a population of primary nucleic acid probes may have 1, 2, 3, 4, 5, 6, 7, 8, 9, 10, 11, 12, 13, 14, 15, 16, 17, 18, 19, 20 etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially producing a relatively large number of nucleic acid probes from a relatively small number of read sequences, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. If each of the primary nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of primary nucleic acid probes, up to 10 probes may be separately identified. For example, one of ordinary skill in the art would understand that, for k read sequences in a population with n read sequences on each probe, up to x different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance, some probes may contain 2 read sequences while other probes may contain 3 read sequences.

In some aspects, the read sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases), when using a pool of primary nucleic acid probes comprising read sequences, wherein each pool each pool of probes encode, via read sequences, a N-bit binary code with a Hamming weight of at least 2 that was assigned to each distinct target nucleic acid (e.g. RNA transcript). Multiple rounds of binding determinations, e.g., using different nucleic acid probes, can then be used to create a “codeword,” e.g., for that spatial location based on binding of the readout probes to the read sequences of the primary probes. In some embodiments, the N-bit binary code may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g., where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a primary nucleic acid probe contains 2 read sequences, then up to 6 primary nucleic acid probes could be identified; but the number of primary nucleic acid probes used may be less than 6. Similarly, for k read sequences in a population with n read sequences on each primary nucleic acid probe, x different probes may be produced, but the number of primary nucleic acid probes that are used may be any number more or less than k. In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2n different targets may be possible for n rounds of probes, but the number of nucleic acid targets that are actually used may be any number less than 2n. For example, if in each round, each target can give more than two possible outcomes, such as being detected in different color channels, more than 2n (e.g. 3n, 4n . . . ) different targets may be possible for n rounds of probes. In some cases, the number of nucleic acid targets that are actually used may be any number less than this number. In addition, these may be randomly assigned, or assigned in specific ways to increase the ability to detect and/or correct errors.

For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. The Hamming weight refers to the distance between the N-bit binary code assigned to a target and thus each pool of primary oligonucleotide probes as encoded via their read sequences. In embodiments a pool of primary probes may have an assigned N-bit binary code with a Hamming weight of at least 4 and a Hamming weight between pools of 4. In that embodiment, errors can both be detected and corrected. In certain cases, the Hamming distance may be at least 2, at least 3, at least 4, at least 5, at least 6, or the like. In addition, in one set of embodiments, the assignments may be formed as a Hamming code, for instance, a Hamming (7, 4) code, a Hamming (15, 11) code, a Hamming (31, 26) code, a Hamming (63, 57) code, a Hamming (127, 120) code, etc. In another set of embodiments, the assignments may form a SECDED code, e.g., a SECDED (8,4) code, a SECDED (16,4) code, a SCEDED (16, 11) code, a SCEDED (22, 16) code, a SCEDED (39, 32) code, a SCEDED (72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, a code with the same error correcting properties of the SECDED code may be formed by using only binary words that contain a fixed number of ‘1’ bits, such as 4, to encode the targets. In another set of embodiments, the assignments may represent a subset of the possible values taken from codes described above for the purpose of addressing asymmetric readout errors. For example, in some cases, a code in which the number of ‘1’ bits may be fixed for all used binary words may eliminate the biased measurement of words with different numbers of ‘1’s when the rate at which ‘0’ bits are measured as ‘1’s or ‘1’ bits are measured as ‘0’s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

The contents of each of the following references are incorporated herein by reference: U.S. Pat. No. 11,098,303, entitled “Systems and Methods for Determining Nucleic Acids”; U.S. Pat. No. 10,240,146, entitled “Probe Library Construction”; US Patent Publ. No. 2019-0264270, entitled “Matrix Imprinting and Clearing”; and US Patent Publ. No. 2022-0064697, entitled “Amplification methods and systems for MERFISH and other applications,” for further discussions of Multiplexed Error-Robust Fluorescence In Situ Hybridization (MERFISH) and its examples (e.g., MERFISH probes described herein, signal amplification, determining nucleic acid probes, creating codewords, and error detection and correction, etc.).

As discussed above, in certain aspects, signaling entities are determined, e.g., to determine nucleic acid probes and/or to create codewords. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for the identified nucleic acid target may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified nucleic acid target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities or targets optically, e.g., using fluorescence microscopy, using radioactivity, via conjugation with suitable chromophores, or the like. For example, various conventional microscopy techniques that may be used in various embodiments of the invention include, but are not limited to, epi-fluorescence microscopy, total-internal-reflectance microscopy, highly inclined thin-illumination (HILO) microscopy, light-sheet microscopy, scanning confocal microscopy, scanning line confocal microscopy, spinning disk confocal microscopy, or other comparable conventional microscopy techniques.

In some embodiments, in situ hybridization (ISH) techniques for labeling nucleic acids such as DNA or RNA may be used, e.g., where nucleic acid probes may be hybridized to nucleic acids in samples. These may be performed, e.g., at cellular-scale or single-molecule-scale resolutions. In some cases, the ISH probes can be composed of RNA, DNA, PNA, LNA, other synthetic nucleotides, or the like, and/or a combination of any of these. The presence of a hybridized probe can be measured, for example, with radioactivity using radioactively labeled nucleic acid probes, immunohistochemistry using, for example, biotin labeled nucleic acid probes, enzymatic chromophore or fluorophore generation using, for example, probes that can bind enzymes such as horseradish peroxidase and approaches such as tyramide signal amplification, fluorescence imaging using nucleic acid probes directly labeled with fluorophores, or hybridization of secondary nucleic acid probes to these primary probes, with the secondary probes detected via any of the above methods.

In some cases, the spatial positions may be determined at super resolutions, or at resolutions better than the wavelength of light or the diffraction limit (although in other embodiments, super resolutions are not required). Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or WO 2013/090360, published Jun. 20, 2013, entitled “High Resolution Dual-Objective Microscopy,” by Zhuang, et al., each incorporated herein by reference in their entireties.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In addition, the signaling entity may be inactivated in some cases. For example, in some embodiments, a first secondary nucleic acid probe containing a signaling entity may be applied to a sample that can recognize a first read sequence, then the first secondary nucleic acid probe can be inactivated before a second secondary nucleic acid probe is applied to the sample. If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion, e.g., by photobleaching the signaling entity, bleaching or chemically altering the structure of the signaling entity, e.g., by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe and washed away via fluid flow.

In some embodiments, various nucleic acid probes (including primary and/or secondary nucleic acid probes) may include one or more signaling entities. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswtichable dyes, photoactivatable dyes, fluorescent dyes, metal nanoparticles, semiconductor nanoparticles or “quantum dots”, fluorescent proteins such as GFP (Green Fluorescent Protein), or photoactivatable fluorescent proteins, such as PAGFP, PSCFP, PSCFP2, Dendra, Dendra2, EosFP, tdEos, mEos2, mEos3, PamCherry, PAtagRFP, mMaple, mMaple2, and mMaple3. Other suitable signaling entities are known to those of ordinary skill in the art. See, e.g., U.S. Pat. No. 7,838,302 or WO2015160690A1, each incorporated herein by reference in its entirety. In some cases, spectrally distinct fluorescent dyes may be used.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl)ethyl, cyclo-dodecyl(dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,+′-dimethoxytrityloxy) butyramidomethyl)phenyl]-ethyl-[2-cyanoethyl-(N,N-diisopropyl)]-phosphoramidite, 1-[2-nitro-5-(6-(N-(4,4′-dimethoxytrityl)-biotinamidocaproamido-methyl)phenyl]-ethyl-[2-cyanoethyl-(N,N-diisopropyl)]-phosphoramidite, or similar linkers. In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl) phosphine, bis(2-mercaptoethyl) sulfone, N,N′-dimethyl-N,N′-bis(mercaptoacetyl) hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, Xmal, PspAI, DpnI, MboI, MnlI, Eco57I, Ksp632I, DraIII, Ahall, Smal, MluI, Hpal, Apal, Bell, BstEII, TaqI, EcoRI, SacI, HindII, HaeII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

Another aspect of the invention is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g., by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

### IV. Kits

The present disclosure provides kits for performing the methods described herein. Kits are provided for preparing biological section samples and determining nucleic acid targets in the sample. In an embodiment, the kit includes primary probes each modified with a moiety that forms a covalent bond with polymer matrix disclosed herein with other components. In certain embodiments, the kit includes a first and/or second anchoring agent disclosed herein together with one or more other components. In an embodiment, the kit also includes labeling agents disclosed herein for identification of cell types or tissue morphology. In some embodiments, the kit includes a number of different labeling agents indicating of a tissue obtained from a particular disease (e.g., solid tumor cancer). In some embodiments, the kit also includes a set of nucleic acid probes (e.g., MERFISH probes described herein) together with one or more other components for MERFISH imaging.

The one or more other kit components can include one or more buffers; a nuclear counterstain; a whole RNA content counterstain; an imaging buffer; software; and other components. A kit can also include instructions for employing the kit components as well as the use of any other reagent not included in the kit. Instructions can include variations that can be implemented.

In certain embodiments provided herein are kit components and protocols for preparing biological section samples for transcriptome analysis. In one embodiment, a kit comprises one or more of the following components: deparaffinization buffer, decrosslinking buffer, conditioning buffer, sample prep wash buffer, formamide wash buffer, gel embedding premix, clearing premix, gel coverslip, pre-anchoring activator, anchoring buffer and digestion premix. Also provided may be anchor probes for immobilizing target nucleic acid (e.g. RNA transcripts) and target probes and reagents thereof used to specifically detect the target nucleic acid in the prepared sample. In embodiments, kits are provided comprising at least primary probes of this disclosure for anchoring in a polymer matrix.

Although embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

It must also be noted that, as used in the specification and the appended claims, the singular forms “a,” “an” and “the” include plural references unless the context clearly dictates otherwise. For example, reference to “a probe” is intended to also include a plurality of probes and reference to “a target” is intended to also include a plurality of targets and the like.

The term “or” is used herein in the inclusive sense, i.e., equivalent to “and/or” unless the context clearly requires otherwise.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following description and appended claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The term “about” or “approximately” means an acceptable error for a particular value as determined by one of ordinary skill in the art, which depends in part on how the value is measured or determined, or a degree of variation that does not substantially affect the properties of the described subject matter.

The use of “comprise”, “comprises”, “comprising”, “contain”, “contains”, “containing”, “include”, “includes”, and “including” is not intended to be limiting, and means that at least the named compound, element, particle, or method step is present in the composition or article or method, but does not exclude the presence of other compounds, materials, particles, method steps, even if the other such compounds, material, particles, method steps have the same function as what is named.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a fabric or system does not preclude the presence of additional components or intervening components between those components expressly identified.

The skilled artisan will understand that the figures, described above, and example, described below, are for illustration purposes only. Neither the figures nor the examples are intended to limit the scope of the disclosed teachings in any way.

### V. EXAMPLES

The following examples are provided to illustrate certain disclosed embodiments and are not to be construed as limiting the scope of this disclosure in any way.

### Example 1. Generation of Primary Nucleic Acid Probes Pools with an Acrydite Moiety

This example illustrates generation of a primary nucleic acid probes and pools thereof to be used with the methods of this disclosure. In embodiments, each primary nucleic acid probe comprises an acrydite moiety (for covalent attachment to a polymer matrix), a target hybridization sequence (for binding to a nucleic acid target in a biological sample) and one or more read sequences (for binding of secondary NA probes with a label for imaging). The primary nucleic acid probes are grouped into pools based on the target hybridization sequence wherein each pool is specific for a distinct nucleic acid target and the read sequences of each pool is decoded to form codewords.

In embodiments, an array-synthesized complex oligo pool, containing approximately thousands to hundreds of thousand sequences, was used as a template for the enzymatic amplification of the primary nucleic acid probe. Each template sequence in the oligo pool contained a central target region (e.g., target hybridization sequence), two flanking read sequences, a forward primer sequence and a T7 promoter sequence. In the first step, the template was amplified through RT-PCR on BioRad CFX96 qPCR machine by mixing the following reagents: 20X EvaGreen® Dye (Biotium 31000), forward primer (IDT, 100 uM), reverse primer (IDT, 100 uM), 100 ng template, nuclease free water and 2X Phusion Hot Start II DNA Polymerase (ThermoFisher F549L). The thermocycling program used for RT-PCR was the following: a) 98° C. for 3 minutes; b) 98° C. for 10 seconds; c) 50° C. for 10 seconds; d) 72° C. for 15 seconds; e) measure fluorescence; f) 72° C. for 10 seconds; g) Repeat steps b through f until the signal amplification rate decreases. The amplified products were then purified through Spin V columns (Zymo, C1012-50) and diluted in water. The RT-PCR product was in vitro transcribed into RNA using a Quick HiScribe T7 Polymerase kit (NEB E2040S) for 12-18 hours, column purified, and the RNA product was resuspended in RNase-free water. The RNA product was reverse-transcribed into DNA using Maxima H Minus Reverse Transcriptase (Thermo Fisher, EP0751) for 1.5 hours at 50° C. The product was treated with pH 9 0.25M NaOH, 0.125M EDTA at 95° C. for 20 minutes, then neutralized with 1M HCL to pH 7, and column purified. In this step, the RNA was reverse transcribed back into DNA via a 5′acrydite-modified forward primer, allowing acrydite modification to be added to each ssDNA. Acrydite is an attachment chemistry based on an acrylic phosphoramidite that can be added to oligonucleotides as a 5′-modification. Acrydite-modified oligonucleotides covalently react with thiol-modified surfaces or can be incorporated into polyacrylamide gels during polymerization. After purification, the product (“primary nucleic acid probe comprising an acrydite moiety”) was then concentrated using a standard ethanol precipitation technique, and the single-stranded DNA pellet was then resuspended in RNase-free water. See FIG. 2.

### Example 2. Anchoring Primary Nucleic Acid Probes in a Polymer Matrix, Clearing Cellular Components (Including the Nucleic Acid Targets) and Imaging-MERFISH Imaging in U2OS Cells and Mouse Brain Tissue Samples

This example illustrates the use of anchored acrydite-modified primary nucleic acid probes (which hybridize with RNA in a biological sample) in a polymer matrix providing for removal of cellular targets including the target RNA (e.g., RNase-insensitive methods to determine RNA in biological samples) according to certain embodiments of the invention. This example demonstrates the anchoring of acrydite-modified MERFISH probes, prepared according to Example 1, subsequent cellular component clearing and imaging. This example is a modified Protocol A, wherein no anchoring agent was added. See FIG. 1. In general, to perform imaging of the anchored MERFISH probes in this example the following steps were performed: oligo probes targeting different RNA species (primary nucleic acid probes of Example 1) were synthesized in silico (step 1) and modified in vitro to create functional moieties (e.g., acrydite moiety) that can bind to a polymer matrix (e.g., hydrogel or polyacrylamide gel). A biological sample (e.g. U2OS cell line and fresh frozen mouse brain samples) was fixed by 4% paraformaldehyde (PFA) for 15 minutes at room temperature and then permeabilized by 70% ethanol overnight at 4° C. After washing the sample briefly with Sample Prep Wash Buffer (Vizgen, 20300001), the acrydite-modified primary probes were diluted in Vizgen's encoding probe hybridization buffer at a final concentration of 2 nanomole per probe (Vizgen 10400001) and added to the biological sample (e.g., cell culture sample or tissue sample) for two days at 37° C. to hybridize with target RNA species in situ. Any unbound primary probes were removed with washing with Vizgen's Formamide Wash Buffer (Vizgen, 20300002) at 47° C. for 30 minutes, twice. Next, the biological sample was embedded in a polymer matrix using the Gel Embedding Premix (Vizgen 20300004), ammonium persulfate (Sigma, 09913-100G) and TEMED (N,N,N′,N′-tetramethylethylenediamine) (Sigma, T7024-25ML) from the MERSCOPE Sample Prep Kit (Vizgen, 10400012) wherein the acrydite moiety of the primary probe formed a covalent bond with the polyacrylamide gel. The gel embedded biological sample was treated with tissue clearing reagents consisting of 50 μL of Protease K (NEB, P8107S) and 5 mL of Clearing Premix (Vizgen 20300003) at 37C overnight to remove cellular components. The anchored primary probes, cleared of cellular components, were imaged using labeled nucleic acid probes (e.g., readout probes) that hybridized with the read sequences of the MERFISH probes, the hybridization and imaging steps repeated until the codeword for each RNA target was decoded. In embodiments, an advantage of this method is that the imaging is insensitive to RNA degradation wherein the hybridization of readout probes and imaging is performed independent of the presence of target RNA (or absence thereof).

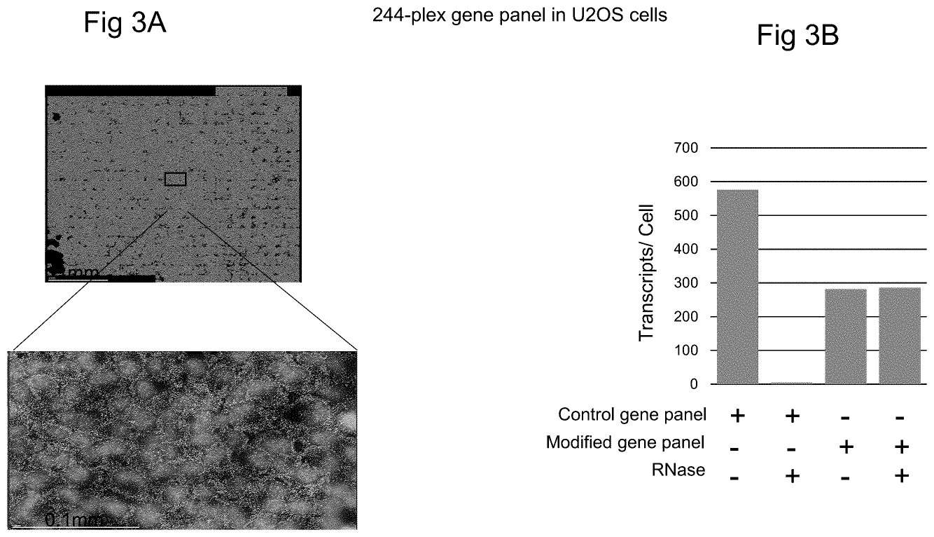

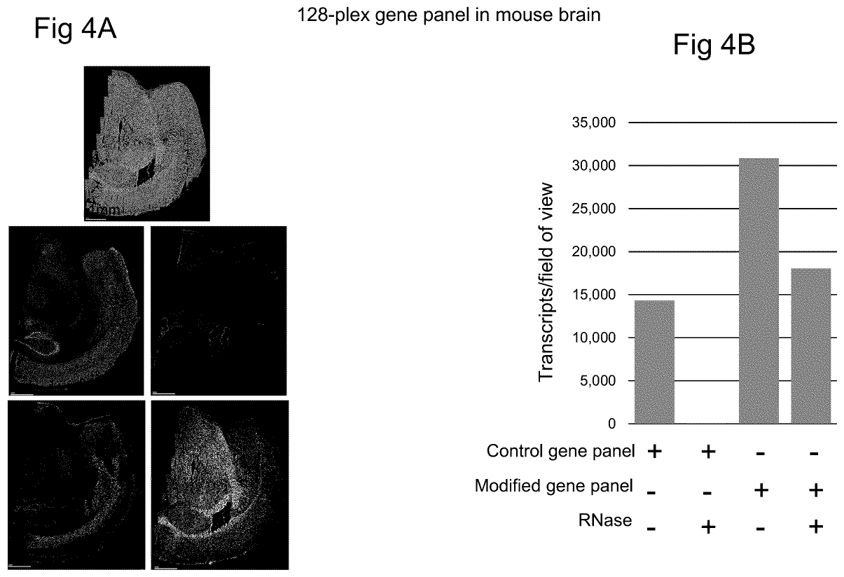

In this example U2OS cell culture or fresh frozen mouse brain samples were hybridized with primary nucleic acid probes with an acrydite moiety and without (control) using a 244-plex gene panel (e.g., 244 primary nucleic acid probe pools) for U2OS samples and a 128-plex gene panel for mouse brain, gel embedded and cleared (with and without RNase H and A to degrade RNA) followed by MERFISH imaging on MERSCOPE for in situ gene expression profiling. The following four conditions were tested: a) control gene panel (e.g., primary probes without the acrydite moiety), no RNase treatment; 2) control gene panel, RNase treatment; 3) modified gene panel (e.g., primary probes comprising an acrydite moiety as generated in Example 1), no RNase treatment; 4) modified gene panel, RNase treatment. See FIGS. 3A and 3B; FIG. 3A shows spatial distribution of all 244 genes in U2OS samples and B) shows RNA transcript counts per cell among the four different conditions. See Figure FIGS. 4A and 4B; FIG. 4A shows spatial distribution of all 128 genes in mouse brain samples and B) shows RNA transcript counts per field of view (200×200 μm) among the four different conditions. This example demonstrates the anchored primary probes (as a proxy for the target RNA in the biological sample) can be imaged after RNase treatment providing an RNase insensitive method for imaging RNA transcripts in a biological sample. In other words, the biological sample was no longer sensitive to RNA degradation and was used for prolonged imaging, since the DNA oligos were immobilized in the gel and not affected by RNA degradation processes in the sample.

### Example 3: Chemical Modification of Primary Nucleic Acid Probes after In Situ Hybridization with Target RNA in a Biological Sample and Anchoring of the Chemically Modified Primary Probes

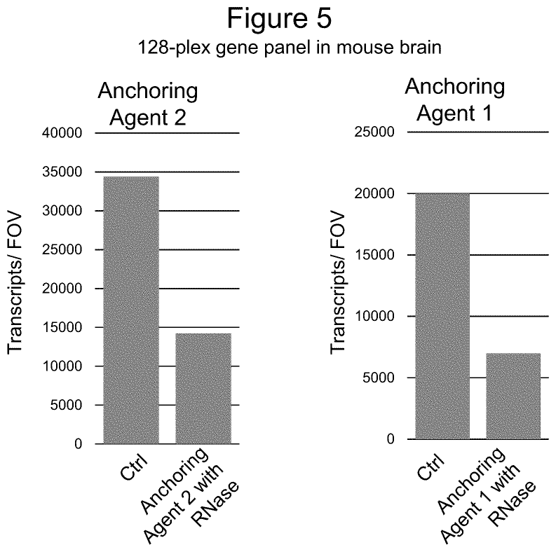

Example 3A: This Example illustrates primary probes (without an acrydite moiety) can be chemically modified in situ using an anchoring agent with a moiety that covalently binds to a polymer matrix. In embodiments, the anchoring agent forms a covalent bond with the primary nucleic acid probe and further comprises an acrydite moiety for covalent attachment to the polymer matrix. In this example, a biological sample (e.g. fresh frozen mouse brain sample) was fixed by 4% paraformaldehyde (PFA) for 15 minutes at room temperature and then permeabilized by 70% ethanol overnight at 4° C. After washing the sample briefly with Sample Prep Wash Buffer (Vizgen, 20300001), the primary probes (a 128-plex gene panel without an acrydite moiety) were diluted in Vizgen's encoding probe hybridization buffer at a final concentration of 2 nanomole per probe (Vizgen 10400001) and added to the biological sample (e.g., cell culture sample or tissue sample) for two days at 37° C. to hybridize with target RNA species in situ. Any unbound primary probes were removed with washing with Vizgen's Formamide Wash Buffer (Vizgen, 20300002) at 47 C for 30 minutes, twice. Then, anchoring agent (anchoring agent 1 or Vizgen pre-anchoring activator (Vizgen, PN 20300113) was added to the hybridized primary probes for anchoring of the primary probes in the polymer matrix at 37° C. for 1 hour. The anchoring agents add acryloyl “tails” to the backbone of DNA or RNA probes. Next, the biological sample was embedded in a polymer matrix using the Gel Embedding Premix (Vizgen 20300004), ammonium persulfate (Sigma, 09913-100G) and TEMED (N,N,N′,N′-tetramethylethylenediamine) (Sigma, T7024-25ML) from the MERSCOPE Sample Prep Kit (Vizgen, 10400012) wherein the acrydite moiety of the primary probe formed a covalent bond with the polyacrylamide gel. The gel embedded biological sample was treated with tissue clearing reagents consisting of 50 μL of Protease K (NEB, P8107S) and 5 mL of Clearing Premix (Vizgen 20300003) at 37C overnight to remove cellular components, followed by treatment with RNase A and H to degrade RNA, and subsequently imaged on MERSCOPE for in situ gene expression profiling. See FIG. 5, which shows the number of imaged transcripts per field of view (FOV) via the primary probes. This example demonstrates the primary probes can be chemically modified in situ to anchor via a covalent bond to the polymer matrix wherein about 30%, as compared to the control, where no anchoring reagent and Rnase treatment were added, were retained in the polymer matrix for MERFISH imaging.

Example 3B: This Example illustrates an additional anchoring strategy for anchoring primary probes (without an acrydite moiety) by anchoring the RNA in a polymer matrix and then chemically modify the primary probes in situ using an anchoring agent with a moiety that covalently binds to a polymer matrix. In embodiments, anchoring the target nucleic acid (e.g., RNA) comprises contacting the biological sample with a first and/or second anchoring agent, wherein the first anchoring agent forms a covalent bond with the target nucleic acid and the second anchoring agent comprises an oligonucleotide that hybridizes with the target nucleic acid.

In this example, mouse brain samples are treated with a first anchoring agent and a second anchoring agent, wherein the first anchoring agent was an alkylating agent derivatized with an acrydite moiety and the second anchoring agent comprised a poly-dT sequence that hybridized to the RNA transcripts and an acrydite moiety. The biological sample was then embedded in a polymer matrix and covalent bonds formed between the polyacrylamide gel and the acrydite moieties of the first and second anchoring agents, and cellular components cleared. Next the primary probes were added to the cleared sample and hybridized with the RNA transcripts corresponding to the 128 genes of the primary probes, the primary probes were chemically modified with either Anchoring agent 1 or Vizgen pre-anchoring activator (Vizgen, PN 20300113), the biological sample then gel embedded with a second gel (to form covalent bonds with the acydite moieties of the anchoring agents), treated with RNase A and H to degrade RNA, and subsequently imaged on MERSCOPE for in situ gene expression profiling.

Specifically, a biological sample (e.g. fresh frozen mouse brain sample) was fixed by 4% paraformaldehyde (PFA) for 15 minutes at room temperature and then permeabilized by 70% ethanol overnight at 4° C. After washing the sample briefly with Sample Prep Wash Buffer (Vizgen, 20300001), the sample was then treated with 5 ml Conditioning Buffer (Vizgen, 20300116) at 37° C. for 30 minutes, 100 μl Pre-Anchoring Reaction Buffer consisting of Conditioning Buffer (Vizgen, 20300116), Pre-Anchoring Activator (Vizgen, 20300113) and RNase inhibitor (NEB, M0314L) at 37° C. for 2 hours, and 100 μl Anchoring Buffer (PN 20300117) at 37° C. overnight. The biological sample was embedded in a polymer matrix using the Gel Embedding Premix (Vizgen 20300004), ammonium persulfate (Sigma, 09913-100G) and TEMED (N,N,N′,N′-tetramethylethylenediamine) (Sigma, T7024-25ML) from the MERSCOPE Sample Prep Kit (Vizgen, 10400012) wherein the acrydite moiety of the primary probe formed a covalent bond with the polyacrylamide gel. The gel embedded biological sample was treated with tissue clearing reagents consisting of 50 μL of Protease K (NEB, P8107S) and 5 mL of Clearing Premix (Vizgen 20300003) at 37C overnight to remove cellular components. Next, the primary probes (a 128-plex gene panel without an acrydite moiety) were diluted in Vizgen's encoding probe hybridization buffer at a final concentration of 2 nanomole per probe (Vizgen 10400001) and added to the biological sample (e.g., cell culture sample or tissue sample) for two days at 37° C. to hybridize with target RNA species in situ. Any unbound primary probes were removed with washing with Vizgen's Formamide Wash Buffer (Vizgen, 20300002) at 47° C. for 30 minutes, twice. Then, anchoring agent (Anchoring agent 1 or Vizgen pre-anchoring activator (Vizgen, PN 20300113) was added to the hybridized primary probes for anchoring of the primary probes in the polymer matrix at 37° C. for 1 hour. The anchoring agents add acryloyl “tails” to the backbone of DNA or RNA probes. The sample was gel embedded again and followed by treatment with RNase A and H to degrade RNA, and subsequently imaged on MERSCOPE for in situ gene expression profiling.

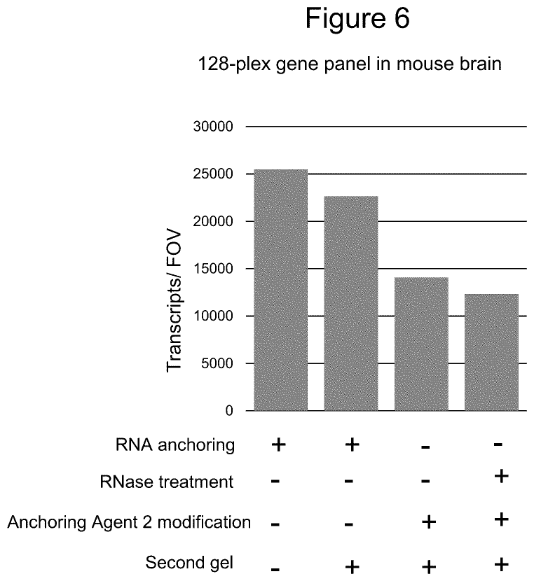

See FIG. 6, which shows the number of imaged transcripts per field of view (FOV) via the primary probes. This example demonstrates the improved efficiency of the anchored primary probes that were chemically modified in situ to anchor via a covalent bond to the polymer matrix, as compared to no RNA anchoring and example 3A, for MERFISH imaging.

### Example 4. Double Modification with 5′ Acrydite-Modified Primary Probe Pool and In Situ Modification with an Anchoring Agent Sufficiently Anchors MERFISH Probes in a Polymer Matrix for MERFISH Imaging of a U20S Cell Culture Sample with a 244-Plex Gene Panel

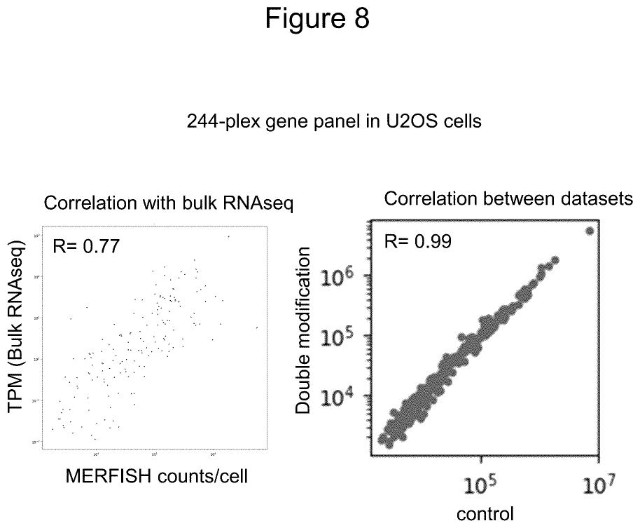

FIG. 1 shows a schematic (“A”) of the experiment workflow for MERFISH imaging of a U2OS cell culture sample with a 244-plex gene panel (“primary probe pools”) using the “double modification” strategy, wherein the biological sample is contacted with a primary nucleic acid probe and an anchoring agent, wherein the primary probe comprises an acrydite moiety and hybridizes with a target nucleic acid and the anchoring agent forms a covalent bond with the primary nucleic acid probe. First, the biological sample was fixed with 4% PFA and permeabilized by 70% ethanol overnight at 4° C. Then introduced into the experiment for hybridization was either the control or acrydite-modified 244-plex MERFISH primary probe pools diluted in Vizgen's encoding probe hybridization buffer at a final concentration of 2 nanomole per probe (Vizgen 10400001) for two days at 37° C. After two washes with Vizgen's Formamide Wash Buffer (Vizgen, 20300002) at +7° C. for 30 minutes each, the samples were either chemically modified with an anchoring agent (either Anchoring agent 1 or Vizgen pre-anchoring activator (Vizgen, PN 20300113) at 37° C. for 1 hour or left untreated. The samples were then gel embedded and cellular components cleared using Vizgen's Sample Preparation Kit (Vizgen, 10400012). One sample was further hybridized with acrydite-modified adapter that can bind to the forward primer overhang of the acrydite-modified library, and gel embedded again with a second gel. Samples were then treated with RNase A and H to degrade RNA, then imaged on MERSCOPE for in situ gene expression profiling. Double modification of the sample resulted in retention of the primary probes for MERFISH imaging and rendered the sample resistant to RNase treatment for MERFISH imaging (repeated rounds of hybridization and imaging). The detection efficiency with double modification is equivalent to the control samples without RNase treatment. See FIG. 7. A correlation analysis with bulk RNAseq data and control sample was performed, which indicated that the MERFISH measurement of the double modified primary probes was accurate and consistent with the MERFISH results in the control experiment. See FIG. 8.

### Example 5. Double Modification with 5′ Acrydite-Modified Primary Probe Pools and In Situ Modification with an Anchoring Agent Sufficiently Anchors MERFISH Primary Probes for MERFISH Imaging of Mouse Brain with a 128-Plex Gene Panel and Two Different Sample Preparation Procedures

FIG. 1 depicts a schematic of the experiment procedure for MERFISH imaging of mouse brain with a 128-plex gene panel and two different sample preparation procedures (“A” and “B”). Briefly, protocol C is the standard MERFISH protocol described by Vizgen's MERSCOPE User Guide for fresh and fixed frozen samples (Vizgen doc 91600002) where samples were hybridized with standard MERFISH encoding probe library, gel embedded and cellular components cleared for imaging. The methodologies developed for removing cellular components from a gel embedded tissue section work sufficiently well with fresh and fixed frozen samples wherein anchoring oligonucleotides and primary probes are added to a sample, then embedded in a gel and the cellular components cleared or removed followed by imaging (“Protocol C”).

For Protocol A the biological sample was contacted with a primary nucleic acid probe and an anchoring agent, wherein the primary probe comprises an acrydite moiety and hybridizes with a target nucleic acid and the anchoring agent forms a covalent bond with the primary nucleic acid probe. That step was followed by embedding the biological sample in a polymer matrix wherein the primary nucleic acid probe and the anchoring agent each formed a covalent bond with the polymer matrix, cellular components cleared from the polymer matrix wherein the primary nucleic acid probe hybridized to the target nucleic acid remains anchored in the polymer matrix, RNase A and H were added to remove the RNA and the immobilized sample imaged.

For Protocol B the RNA in the biological sample was anchored in a polymer matrix, wherein a first and second anchoring agent was added to the biological sample, wherein the first anchoring agent forms a covalent bond with the target nucleic acid and the second anchoring agent comprises an oligonucleotide that hybridizes with the target nucleic acid. Specifically, the first anchoring agent was an alkylating agent derivatized with an acrydite moiety and the second anchoring agent comprises a poly-d′T portion that hybridizes to the target nucleic acid and an acyrdite moiety for covalently attaching to the polymer matrix. That step was followed by embedding the biological sample in a polymer matrix wherein the first and second anchoring agent each formed a covalent bond with the polymer matrix, cellular components cleared from the polymer matrix wherein the target RNA remained anchored in the polymer matrix. Next the anchored target nucleic acid was contacted with a primary nucleic acid probe and a first anchoring agent, wherein the primary nucleic acid probe comprises an acrydite moiety and hybridized with the anchored target RNA and the anchoring agent formed a covalent bond with the primary nucleic acid probe, a second gel embedding step was performed wherein the primary nucleic acid probe and the anchoring agent each formed a covalent bond with the polymer matrix, Rnase A and H were added to remove the RNA and the immobilized sample imaged.

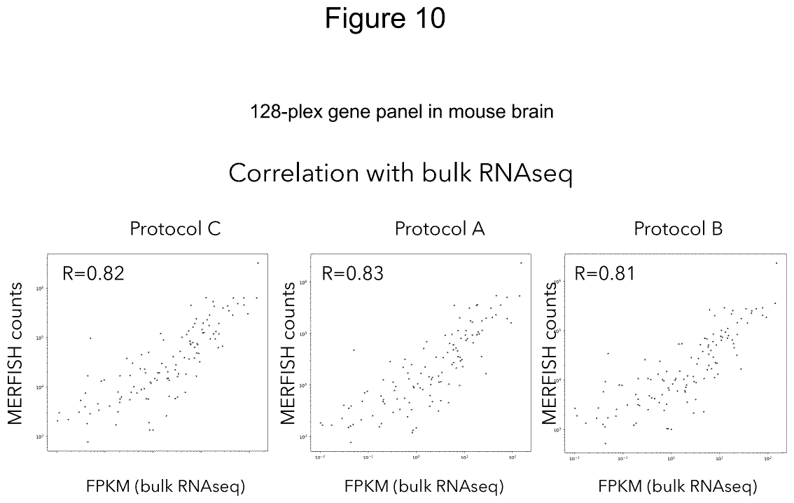

Double modification resulted in the retention of encoding probes for MERFISH imaging and rendered the sample resistant to RNase treatment for MERFISH imaging. Detection efficiency with double modification was equivalent to the control samples without RNase treatment. See FIG. 9. A correlation analysis was performed with bulk RNAseq data and control sample, indicating the measurement of MERFISH signal with the double-modification was accurate and consistent with the MERFISH results in the control experiment. See FIG. 10.

1. A method of anchoring a primary nucleic acid probe within a matrix and clearing cellular components comprising:
a. contacting a biological sample with a primary nucleic acid probe and an anchoring agent, wherein the primary probe comprises an acrydite moiety and hybridizes with a target nucleic acid and the anchoring agent forms a covalent bond with the primary nucleic acid probe;
b. embedding the biological sample in a polymer matrix wherein the primary nucleic acid probe and the anchoring agent each form a covalent bond with the polymer matrix; and
c. clearing cellular components from the polymer matrix wherein the primary nucleic acid probe hybridized to the target nucleic acid remains anchored in the polymer matrix to form a matrix anchored primary nucleic acid probe sample.

2. The method of claim 1, further comprising adding RNase to the matrix anchored primary probe sample and clearing the target nucleic acid from the polymer matrix.

3. The method of claim 1, wherein the anchoring agent is an alkylating agent.

4. The method of claim 3, wherein the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin.

5. The method of claim 1, wherein the anchoring agent and the primary nucleic acid probe each comprise an acrydite moiety that covalently binds the polymer matrix.

6. The method of claim 1, wherein the anchoring agent is an alkylating agent derivatized with an acrydite moiety.

7. The method of claim 1, wherein the target nucleic acid is RNA.

8. The method of claim 1, wherein the target nucleic acid is DNA.

9. The method of claim 1, wherein the primary nucleic acid probe comprises a target sequence that hybridizes to the target nucleic acid, one or more read sequences and an acrydite moiety that covalently binds the polymer matrix.

10. The method of claim 9, wherein the primary nucleic acid probes are single molecule (sm) FISH probes or multiplexed error robust fluorescence in situ hybridization (MERFISH) probes.

11. The method of claim 10, further comprising determining read sequences based on contacting the primary nucleic acid probe with a plurality of secondary nucleic acid probes comprising a recognition sequence that hybridizes to the read sequence of the primary nucleic acid probe.

12. The method of claim 2, further comprising imaging using multiplexed fluorescence in situ hybridization comprising one or more sequential steps of adding a plurality of secondary nucleic acid probes comprising a label moiety that hybridize to the anchored primary nucleic acid probes.

13. (canceled)

14. The method of claim 1, wherein step a) comprises contacting the biological sample comprising a plurality of distinct RNA species with a plurality of primary nucleic acid probe pools each of the nucleic acid probes comprising a target sequence, one or more read sequences and an acrydite moiety that covalently binds the polymer matrix, wherein each pool of nucleic acid probes hybridize to a distinct RNA species and each pool of probes encode a N-bit code that was assigned to each distinct RNA species, wherein each assigned N-bit code is a valid codeword with a Hamming distance of 2 between valid codewords.

15-48. (canceled)

49. A method for imaging a nucleic acid target within a matrix and clearing cellular components comprising:
a. contacting a biological sample with a primary nucleic acid probe and an anchoring agent, wherein the primary probe comprises an acrydite moiety and hybridizes with a target nucleic acid and the anchoring agent forms a covalent bond with the primary nucleic acid probe;
b. embedding the biological sample in a polymer matrix wherein the primary nucleic acid probe and the anchoring agent each form a covalent bond with the polymer matrix;
c. clearing non-target cellular components from the polymer matrix wherein the primary nucleic acid probe hybridized to the target nucleic acid remains anchored in the polymer matrix to form a matrix anchored primary nucleic acid probe sample; and,
d. contacting the anchored primary nucleic acid probe with a plurality of secondary nucleic acid probes comprising a fluorescent label and a recognition sequence that hybridizes to a sequence of the primary nucleic acid probe and imaging the target nucleic acids.

50-156. (canceled)

157. A plurality of primary nucleic acid probe pools each of the primary nucleic acid probes comprising: a target sequence, one or more read sequences and an acrydite moiety, wherein each pool of primary nucleic acid probes is configured to hybridize to a distinct RNA species in a sample and each pool of primary probes encode a N-bit code that was assigned to each distinct RNA species, wherein each assigned N-bit code is a valid codeword.

158-167. (canceled)

168. The primary probe pools of claim 157, wherein the primary nucleic acid probes are covalently bonded to an anchoring agent.

169. The primary probe pools of claim 168, wherein the anchoring agent is an alkylating agent.

170. The primary probe pools of claim 169, wherein the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin.

171. The primary probe pools of claim 169, wherein the anchoring agent and the primary nucleic acid probes each comprise an acrydite moiety configured to covalently bind a polymer matrix.

172. The primary probe pools of claim 168, wherein the anchoring agent is an alkylating agent derivatized with an acrydite moiety.

173-184. (canceled)
